(19) United States
(12) Patent Application Publication
(10) Pub. No.: US 2016/0215103 A1
(43) Pub. Date: Jul. 28, 2016

(54) SYNTHESIS OF SILK FIBROIN MICRO- AND SUBMICRON SPHERES USING A CO-FLOW METHOD

(71) Applicant: TUFTS UNIVERSITY, Medford, MA (US)

(72) Inventors: Fiorenzo Omenetto, Lexington, MA (US); Giovanni Perotto, Cambridge, MA (US); Benedetto Marelli, Somerville, MA (US); David Kaplan, Concord, MA (US); Alexander Mitropoulos, Winchester, MA (US)

(21) Appl. No.: 15/025,140

(22) PCT Filed: Sep. 26, 2014
(86) PCT No.: PCT/US14/57701

§ 371 Date: Mar. 25, 2016

Related U.S. Application Data
(60) Provisional application No. 61/883,933, filed on Sep. 27, 2013.

(51) Int. Cl. C08J 3/12; C07K 1/02; C07K 14/435

(52) U.S. Cl. CPC C08J 3/12; C07K 14/43504; C07K 1/02; C08J 2389/00

(57) ABSTRACT

This application relates to silk fibroin particles that are structurally uniform. Related methods are also disclosed.

### GOVERNMENT SUPPORT

This invention was made with government support under grant W911NF-11-1-0254 awarded by the Defense Advanced Research Projects Agency (DARPA). The U.S. government has certain rights in the invention.

### RELATED APPLICATIONS

The subject matter of this application relates to U.S. provisional application concurrently filed herewith, entitled “LOW MOLECULAR WEIGHT SILK FIBROIN AND USES THEREOF”, the entire contents of which are incorporated herein by reference.

### BACKGROUND

Microfluidic devices have gained popularity in recent years due to their ease of fabrication and handling of small volumes of liquids, making them useful for high-throughput applications. However, the ability to control particle size distribution on the micron scale and sub-micron (e.g., nano) scale remains a challenge in these contexts, particularly with respect to reproducibility of obtaining biopolymer-based nanoparticles.

### SUMMARY OF THE INVENTION

Among other things, the present invention encompasses the recognition that it is possible to generate silk fibroin particles by controlling fluid interactions. More specifically, the present application describes methods by which droplets of a silk fibroin solution are deposited into an immiscible solution to form silk fibroin spheres of micro- and nano-scales, and the formation of such silk fibroin spheres is mediated at least in part via controlled interactions between the two fluids (i.e., the silk fibroin solution and the immiscible solution).

In particular, the process does not require the silk fibroin solution to be pre-blended with another polymer material; therefore, the resulting micro- and nano-particles are made essentially of silk fibroin and water, without contamination. The resulting silk fibroin particles are characterized, for example, by their substantially spherical shape, small size (micron to sub-micron range), structurally uniform particles within a pool or population of silk fibroin particles, and smooth surface morphology (e.g., texture). Fluid dynamics-based methods described herein, which are broadly referred to as the “co-flow” technique, allow the generation of substantially uniform silk fibroin spheres that are essentially free of contaminations from co-polymer materials, which were required in previously described methods.

Furthermore, the present invention includes the discovery that silk fibroin particles having certain desirable characteristics can be produced by selectively varying the molecular weight (e.g., fragment sizes) of silk fibroin polypeptides. In particular, low molecular weight silk fibroin polypeptides are useful for producing sub-micron (i.e., nano) range particles by the methods described herein. Moreover, as described in further detail herein, this and related techniques do not require that silk fibroin particles (e.g., spheres) be crosslinked with the use of a crosslinking agent, such as alcohols, in the process, providing added flexibility for a wide range of downstream applications.

Provided techniques may be readily adaptable to provide inexpensive, user-friendly formats, such as microfluidic systems. Microfluidic systems have a small footprint and are easily scalable providing cost effective, small, reproducible devices. Such devices have been the subject of significant research to produce consistent droplets (and spheres) exploiting fluid hydrodynamics. In particular, capillary devices may be successfully employed to generate controlled emulsions because of the dependence of the droplet size on the flow rates of immiscible fluids.

The present application now describes enabling adaptations of such devices for the synthesis of biopolymer-based monodispersed particles on the micron- and submicron scales that can be used, among other things, for drug delivery, sensing applications (e.g., magnetic particles), contrast agents in MRI, and core/shell nanosphere technologies.

### BRIEF DESCRIPTION OF THE DRAWING

FIG. 1 provides a schematic diagram of an exemplary co-flow capillary device fabricated from stainless steel components. The dark spheres represent the discrete phase in the continuous PVA phase. b) Particle distribution after purification as measured by DLS. The sample was synthesized with 60 mg/ml of 60 minute boiled silk, and a flow rate ratio of 10. c) A SEM image of a collection of dried microspheres of the same sample. Scale bar is 10 μm.

FIG. 2 provides SEM images of silk spheres synthesized by changing PVA concentrations: a) 5%, b) 4%, and c) 2%. Scale bars are 2 μm. d) Average sphere diameter measured by DLS as a function of PVA concentration. Bars represent variance.

FIG. 3 provides SEM images of silk spheres synthesized by changing silk concentrations: a) 60 mg/ml, b) 30 mg/ml, and c) 10 mg/ml concentration. Scale bars are 2 nm. The continuous phase was 5% PVA and the flow rate ratio was 10. d) Average sphere diameter measured by DLS as a function of silk concentration. Inset of the figure shows mass yield at different concentrations. Bars represents variance.

FIG. 4 provides two graphs depicting average diameter of particles as a function of flow rate: a) Average diameter of particles for 10 mg/ml, 30 mg/ml, and 60 mg/ml solutions as functions of the flow rate ratio for 60 minute boil silk. b) Average diameter of particles for 10 mg/ml, 30 mg/ml, and 60 mg/ml solutions as functions of the flow rate ratio for 30 minute boil silk.

FIG. 5 shows cumulative release kinetics for silk spheres. Silk spheres showed a burst release of the drug over the first 24 hrs followed by a steady release. Smaller spheres (blue triangles) showed a faster released compared to bigger spheres (black squares). Inset of the figure shows the correlation between the cumulative release at 168 hrs and the surface to volume ratio.

FIG. 6. Viscosity measurements using a Brookfield viscometer. Shear stress vs. shear rate plots for 30 and 60 minute boil times for 60 mg/ml solutions (a-b), 30 mg/ml solutions (c-d), and 10 mg/ml solutions (e-f). g) Calculated plastic viscosities for 30 minute and 60 minute boiled silk at different concentrations. All data was collected twice.

FIG. 7. Excitation and emission spectra of FITC-BSA loaded silk microspheres.

FIG. 8. Self deconvolved FTIR spectrum of a collection of silk particles.

### DETAILED DESCRIPTION OF CERTAIN EMBODIMENTS

The ability to control particle size distribution (e.g., uniformity) on the micron scale and sub-micron (e.g., nano) scale is important in a number of contexts. For example, for downstream applications involving medical or clinical use, such as drug delivery applications, it is crucial to ensure reproducibility of release kinetics. Generally, monodisperse particles provide greater degree of uniformity in release kinetics than polydisperse particles. Similarly, it is of particular interest to have the ability to finely control particle size and distributions in a number of optical applications involving the use of nano-sized particles, such as the plasmonic resonance of core/shell nanospheres and their tunable, size-dependent, optical properties. Moreover, the use of protein-based materials provides additional utility for these systems, since they can be absorbed by the body with safe degradation.

Silk fibroin, the natural protein extracted from the caterpillar Bombxy mori, in particular, is an appealing biopolymer material due to its unique attributes, such as its natural self-assembly property, ease of conforming to nanoscale sizes, biocompatibility, lack of toxicity, lack of immunogenicity, to name a few. Silk has been shown to maintain the function of entrained biological dopants, allowing silk to act as a carrier for the preservation and delivery of drugs. Moreover it is possible to control the kinetics of silk degradation by managing the degree of crystallinity, thereby regulating the breakdown of the material which, for drug delivery applications, allows for controllable release kinetics of encapsulated drugs in contrast to bulk release.

As such, much effort has been made into developing a technique to reliably produce silk-based nanoparticles with superior uniformity. For example, WO 2011/041395 A2 (PCT/US2010/050698) describes silk-based particles prepared from a co-polymer mixture of silk fibroin and polyvinyl alcohol (PVA), the contents of which are incorporated herein by reference. In that publication, the co-polymer mixture is solidified into a film, mechanically grinded or crushed into a powder-like form, then the PVA is washed away, so as to obtain a product that is predominantly silk-based particles. However, particles prepared by such methods are typically not uniform in size, and also tended to contain a residual unwanted component.

The present application describes a much different approach in achieving the production of improved (e.g., reliably uniform and pure) silk fibroin particles. Moreover, methods described herein eliminates certain steps of production that were necessary in the prior methods, simplifying the process of manufacture. Specifically, the present invention encompasses the recognition that fluid dynamics can be exploited to control fine tuning of silk fibroin particle properties. More specifically, the present invention includes the use of co-flow systems in achieving such effects.

In a general sense, the invention contemplates fluid-fluid interactions, in which two immiscible solutions, namely, so-called “continuous” and “discrete” phases, are contacted with each other under a set of controlled conditions, which facilitates the formation of small droplets of discrete phase solution deposited into the surrounding continuous phase solution. Through the fluid interactions, such droplets made from the discrete phase form small particles, which then can be readily collected by any suitable collection means. For example, a silk fibroin solution can be used as a suitable discrete phase in such methods, while an immiscible solutions, such as PVA, may be used as suitable continuous phase.

Without wishing to be bound by a particular theory, particle (e.g., sphere) generation occurs by break-off of the discrete phase in the bulk fluid stream leading to the formation of monodispersed droplets. Self-assembly of silk fibroin in the droplets facilitates the production of consistently sized spheres in the micron or submicron range.

As already stated, a silk fibroin particle (e.g., silk fibroin sphere) is formed from a droplet of a silk fibroin solution at least in part through its direct contact with an immiscible solution into which the droplet is deposited. It is believed that the formation of such a silk fibroin particle involves condensation, i.e., transfer of water molecules from the silk droplet to the immiscible solution at its interface, without true mixing of the two fluids. Therefore, the silk fibroin particle formed in this way is essentially free of the immiscible solution.

The type of break-off of the droplet may be at least in part determined by the volumetric flow rates of the continuous and discrete flows of the two immiscible fluids, the interfacial tension, and viscosity of the fluids. This in turn determines the size of the droplets that break-off from the initial stream.

Two droplet forming regimes are known in co-flow devices: dripping and jetting. Dripping is based on the difference between viscous drag forces and the surface tension holding the drop to the bulk fluid stream, while jetting is caused by the Rayleigh-Plateau instability within the inner stream. Work described herein indicates that silk submicron spheres are generated in the jetting regime.

Devices operating in the jetting regime have been shown to generate smaller diameter particles compared to the dimensions of the outlet orifice, in agreement with what is observed here. In this case, both fluids are aqueous solutions with low interfacial tension that leads to a reduction in the driving force for liquid jets to break-off into droplets. Once the droplet of the discrete phase is formed silk will condense due in part to its immiscibility with a surrounding immiscible solution. Since the droplet diameter can be controlled by changing fluid parameters like flow rate and viscosity, spheres with controlled and tunable size can be synthesized.

### A Silk Fibroin Particle

Accordingly, “a silk fibroin particle” described herein is a substantially spherical particle, typically in a solid form. As used herein, “substantially spherical” means that a particle is fundamentally or markedly round in shape such that the radius or the broadest width measured from any angle of the particle is identical or close to identical, with relative distortion, for example, of less than 20%, less than 15%, less than 10%, less than 5%, less than 4%, less than 3%, less than 2%, or less than 1% deviation.

Provided silk fibroin particles described herein typically have diameters in micron- to sub-micron-ranges, e.g., ranging between about less than 100 nanometers to several micrometers. Throughout this disclosure, terms such as “sub-micron-” and “nano-” are used interchangeably to refer to particle dimensions generally smaller than one micron (1.0 μm) in diameter, such as a fraction of a micron.

The dimension (typically measured or expressed, for example, as diameter) of such silk fibroin particle can be controlled by varying parameters, such as the volume of a silk fibroin solution droplet from which a silk fibroin sphere forms, viscosity of the silk fibroin solution, molecular weight of silk fibroin polypeptides that make up such a solution, among other factors, as discussed further below. As compared to silk-based particles described in previously disclosed methods, silk fibroin particles of the present invention are generally smaller, can be varied with control, and reliably more uniform.

As discussed in detail below, because individual droplets are formed one at a time under predetermined conditions, it is possible to generate a uniform population of silk fibroin particles using the technique described herein.

Silk fibroin particles described herein are characterized by having low porosity (i.e., a measure of the void spaces in a material and is typically expressed as the ratio of pore volume to its total volume), relative to silk fibroin particles prepared by previously described methods such as the silk-PVA co-polymer method. In that case, the two fluids (i.e., silk fibroin solution and the PVA solution) are first blended to form a co-polymer solution, which is then processed further to create solidified particles. The PVA portion is subsequently washed away, leaving behind predominantly silk-based particles having higher porosity caused by the removal of PVA. In comparison, silk fibroin particles described herein do not depend on the use of a co-polymer blend and therefore do not require the step of removing the second polymer from the particles. As a result, the particles can be made to contain low porosity.

In some embodiments, silk fibroin particles of the present invention have no more than about 20% porosity, e.g., no more than 19%, no more than 18%, no more than 17%, no more than 16%, no more than 15%, no more than 14%, no more than 13%, no more than 12%, no more than 11%, 10%, no more than 9%, no more than 8%, no more than 7%, no more than 6%, no more than 5%, no more than 4%, no more than 3%, no more than 2%, no more than 1%, no more than 0.5%, no more than 0.1% porosity.

Silk fibroin particles described herein are characterized by having a smooth surface morphology, particularly as compared to silk fibroin particles generated by the silk-PVA blend method mentioned above.

Surface smoothness or surface roughness is a measure of the texture of a surface and may be quantified by the vertical deviations of a real surface from its ideal form. If these deviations are large, the surface is rough; if they are small the surface is smooth. The degree of smoothness/roughness can be therefore expressed, for example, as the root mean square (RMS), which is also known as the quadratic mean.

In some embodiments, silk fibroin particles of the present invention have surface roughness lower than 50 nm as measured in RMS, for example, lower than 45 nm, lower than 40 nm, lower than 35 nm, lower than 30 nm, lower than 25 nm, lower than 20 nm, lower than 15 nm, lower than 10 nm, etc.

Silk fibroin particles described herein are substantially free from contaminants, such as a secondary polymer solution typically used as a blending material during manufacture in previously described methods. Accordingly, methods provided herein allow the production of small, pure silk fibroin spheres that consist virtually of silk fibroin protein (i.e., silk fibroin polypeptides) and water. Although the formation of such silk fibroin particles described herein involves direct contact with an immiscible solutions (e.g., a secondary polymer material; see below for further detail) on the external surface, the immiscible solution does not penetrate within the silk fibroin particles themselves.

Water contents in the silk fibroin particles described herein vary, depending on a particular application for which the particles are prepared. Typically, silk fibroin particles described herein have water contents ranging between about 1% and about 75%. For example, in some embodiments, silk fibroin particles described herein have water contents ranging between 5% and 50%, between 5% and 45%, between 5% and 40%, between 5% and 35%, between 5% and 30%, between 5% and 25%, between 5% and 20%, between 5% and 15%, between 5% and 10%, and so on, as measured by weight.

As reported previously, silk fibroin has an inherent self-assembly property. Silk fibroin particles described herein may contain a range of degrees of crystallinity. For example, provided silk fibroin particles may contain a beta-sheet content ranging between about 10% and 70%, e.g., about 10%, about 15%, about 20%, about 25%, about 30%, about 35%, about 40%, about 45%, about 50%, about 55%, about 60%, about 65% or about 75%.

As used herein, the term “silk fibroin” useful for carrying out the present invention includes silkworm fibroin and insect or spider silk protein. See e.g., Lucas et al., 13 Adv. Protein Chem. 107 (1958). For example, silk fibroin useful for the present invention may be that produced by a number of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

In general, silk for use in accordance with the present invention may be produced by any such organism, or may be prepared through an artificial process, for example, involving genetic engineering of cells or organisms to produce a recombinant silk fibroin polypeptide and/or chemical synthesis. In some embodiments of the present invention, silk is produced by the silkworm, Bombyx mori.

As is known in the art, silks are modular in design, with large internal repeats flanked by shorter (˜100 amino acid) terminal domains (N and C termini). Naturally occurring silk fibroin polypeptides have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules. An exemplary list of silk-producing species and corresponding silk proteins may be found in International Patent Publication Number WO 2011/130335, the entire contents of which are incorporated herein by reference.

Cocoon silk produced by the silkworm, Bombyx mori, is of particular interest because it offers low-cost, bulk-scale production suitable for a number of commercial applications, such as textile. Silkworm cocoon silk contains two structural proteins, the fibroin heavy chain (˜350 k Da) and the fibroin light chain (˜25 k Da), which are associated with a family of nonstructural proteins termed sericin, which glue the fibroin brings together in forming the cocoon. The heavy and light chains of fibroin are linked by a disulfide bond at the C-terminus of the two subunits (Takei, F., Kikuchi, Y., Kikuchi, A., Mizuno, S. and Shimura, K. (1987) J. Cell Biol., 105, 175-180; Tanaka, K., Mori, K. and Mizuno, S. (1993) J. Biochem. (Tokyo), 114, 1-4; Tanaka, K., Kajiyama, N., Ishikura, K., Waga, S., Kikuchi, A., Ohtomo, K., Takagi, T. and Mizuno, S. (1999) Biochim. Biophys. Acta, 1432, 92-103; Y Kikuchi, K Mori, S Suzuki, K Yamaguchi and S Mizuno, Structure of the Bombyx mori fibroin light-chain-encoding gene: upstream sequence elements common to the light and heavy chain, Gene 110 (1992), pp. 151-158). The sericins are a high molecular weight, soluble glycoprotein constituent of silk which gives the stickiness to the material. These glycoproteins are hydrophilic and can be easily removed from cocoons by boiling in water. This process is often referred to as “degumming.”

As used herein, the term “silk fibroin” embraces silk fibroin protein, whether produced by silkworm, spider, or other insect, or otherwise generated (Lucas et al., Adv. Protein Chem., 13: 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

Thus, in some embodiments, a silk solution is used to fabricate compositions of the present invention contain fibroin proteins, essentially free of sericins. Provided silk fibroin particles contemplated herein are essentially free of sericins, unless otherwise explicitly specified. “Essentially free of sericins” means that such compositions contain no (e.g., undetectable) or little (i.e., trace amount) sericin such that one of ordinary skill in the pertinent art will consider negligible for a particular use.

In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins. In certain embodiments, silk solutions used to fabricate various compositions of the present invention comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds.

Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Ala-rich” hydrophobic blocks are typically separated by segments of amino acids with bulky side-groups (e.g., hydrophilic spacers).

In some embodiments, core repeat sequences of the hydrophobic blocks of fibroin are represented by the following amino acid sequences and/or formulae:

(SEQ ID NO: 1)
(GAGAGS)₅₋₁₅;
(SEQ ID NO: 2)
(GX)₅₋₁₅
(X = V, I, A);
(SEQ ID NO: 3)
GAAS;
(SEQ ID NO: 4)
(S₁₋₂A₁₁₋₁₃);
(SEQ ID NO: 5)
GX₁₋₄ GGX;
(SEQ ID NO: 6)
GGGX
(X = A, S, Y, R, D V, W, R, D);
(SEQ ID NO: 7)
(S1-2A1-4)₁₋₂;
(SEQ ID NO: 8)
GLGGLG;
(SEQ ID NO: 9)
GXGGXG
(X = L, I, V, P);
GPX
(X = L, Y, I);
(SEQ ID NO: 10)
(GP(GGX)₁₋₄Y)n
(X = Y, V, S, A);
(SEQ ID NO: 11)
GRGGAn;
GGXn
(X = A, T, V, S);
(SEQ ID NO: 12)
GAG(A)₆₋₇GGA;
and
(SEQ ID NO: 13)
GGX GX GXX
(X = Q, Y, L, A, S, R).

In some embodiments, a fibroin peptide contains multiple hydrophobic blocks, e.g., 3, 4, 5, 6, 7, 8, 9, 10, 11, 12, 13, 14, 15, 16, 17, 18, 19 and 20 hydrophobic blocks within the peptide. In some embodiments, a fibroin peptide contains between 4-17 hydrophobic blocks. In some embodiments of the invention, a fibroin peptide comprises at least one hydrophilic spacer sequence (“hydrophilic block”) that is about 4-50 amino acids in length. Non-limiting examples of the hydrophilic spacer sequences include:

(SEQ ID NO: 14)
TGSSGFGPYVNGGYSG;
(SEQ ID NO: 15)
YEYAWSSE;
(SEQ ID NO: 16)
SDFGTGS;
(SEQ ID NO: 17)
RRAGYDR;
(SEQ ID NO: 18)
EVIVIDDR;
(SEQ ID NO: 19)
TTIIEDLDITIDGADGPI
and
(SEQ ID NO: 20)
TISEELTI.

In certain embodiments, a fibroin peptide contains a hydrophilic spacer sequence that is a derivative of any one of the representative spacer sequences listed above. Such derivatives are at least 75%, at least 80%, at least 85%, at least 90%, or at least 95% identical to any one of the hydrophilic spacer sequences.

In some embodiments, a fibroin peptide suitable for the present invention contains no spacer.

As noted, silks are fibrous proteins and are characterized by modular units linked together to form high molecular weight, highly repetitive proteins. These modular units or domains, each with specific amino acid sequences and chemistries, are thought to provide specific functions. For example, sequence motifs such as poly-alanine (polyA) and polyalanine-glycine (poly-AG) are inclined to be beta-sheet-forming; GXX motifs contribute to 31-helix formation; GXG motifs provide stiffness; and, GPGXX (SEQ ID NO: 22) contributes to beta-spiral formation. These are examples of key components in various silk structures whose positioning and arrangement are intimately tied with the end material properties of silk-based materials (reviewed in Omenetto and Kaplan (2010) Science 329: 528-531).

It has been observed that the beta-sheets of fibroin proteins stack to form crystals, whereas the other segments form amorphous domains. It is the interplay between the hard crystalline segments, and the strained elastic semi amorphous regions, that gives silk its extraordinary properties. Non-limiting examples of repeat sequences and spacer sequences from various silk-producing species are provided in An exemplary list of hydrophobic and hydrophilic components of fibroin sequences may be found in International Patent Publication Number WO 2011/130335, the entire contents of which are incorporated herein by reference.

The particular silk materials explicitly exemplified herein were typically prepared from material spun by silkworm, B. Mori. Typically, cocoons are boiled for a suitable duration of time, such as ˜30 minutes or longer, in an aqueous solution of 0.02M Na₂CO₃, then rinsed thoroughly with water to extract the glue-like sericin proteins. The extracted silk is then dissolved in LiBr (such as 9.3 M) solution at room temperature, yielding a 20% (wt.) solution. The resulting silk fibroin solution can then be further processed for a variety of applications as described elsewhere herein. Those of ordinary skill in the art understand other sources may also be appropriate.

The complete sequence of the Bombyx mori fibroin gene has been determined (C.-Z Zhou, F Confalonieri, N Medina, Y Zivanovic, C Esnault and T Yang et al., Fine organization of Bombyx mori fibroin heavy chain gene, Nucl. Acids Res. 28 (2000), pp. 2413-2419). The fibroin coding sequence presents a spectacular organization, with a highly repetitive and G-rich (˜45%) core flanked by non-repetitive 5′ and 3′ ends. This repetitive core is composed of alternate arrays of 12 repetitive and 11 amorphous domains. The sequences of the amorphous domains are evolutionarily conserved and the repetitive domains differ from each other in length by a variety of tandem repeats of subdomains of ˜208 bp.

The silkworm fibroin protein consists of layers of antiparallel beta sheets whose primary structure mainly consists of the recurrent amino acid sequence (Gly-Ser-Gly-Ala-Gly-Ala)n (SEQ ID NO: 21). The beta-sheet configuration of fibroin is largely responsible for the tensile strength of the material due to hydrogen bonds formed in these regions. In addition to being stronger than Kevlar, fibroin is known to be highly elastic. Historically, these attributes have made it a material with applications in several areas, including textile manufacture.

Fibroin is known to arrange itself in three structures at the macromolecular level, termed silk I, silk II, and silk III, the first two being the primary structures observed in nature. The silk II structure generally refers to the beta-sheet conformation of fibroin. Silk I, which is the other main crystal structure of silk fibroin, is a hydrated structure and is considered to be a necessary intermediate for the preorganization or prealignment of silk fibroin molecules. In the nature, silk I structure is transformed into silk II structure after spinning process. For example, silk I is the natural form of fibroin, as emitted from the Bombyx mori silk glands. Silk II refers to the arrangement of fibroin molecules in spun silk, which has greater strength and is often used commercially in various applications. As noted above, the amino-acid sequence of the β-sheet forming crystalline region of fibroin is dominated by the hydrophobic sequence. Silk fiber formation involves shear and elongational stress acting on the fibroin solution (up to 30% wt/vol.) in the gland, causing fibroin in solution to crystallize. The process involves a lyotropic liquid crystal phase, which is transformed from a gel to a sol state during spinning—that is, a liquid crystal spinning process. Elongational flow orients the fibroin chains, and the liquid is converted into filaments.

Silk III is a newly discovered structure of fibroin (Valluzzi, Regina; Gido, Samuel P.; Muller, Wayne; Kaplan, David L. (1999). “Orientation of silk III at the air-water interface”. International Journal of Biological Macromolecules 24: 237-242). Silk III is formed principally in solutions of fibroin at an interface (i.e. air-water interface, water-oil interface, etc.).

The present invention encompasses the recognition that the molecular weight or range of molecular weights of silk fibroin used to prepare silk fibroin particles (e.g., spheres) described herein influences the structural parameters or features of resulting silk fibroin particles. Thus, by controlling the molecular weight of silk fibroin in a silk fibroin solution, it is possible to produce silk fibroin particles having certain desired features. In particular, as described herein, the average particle size and/or particle distribution within a population of silk fibroin particles may be significantly affected by varying molecular weights of silk fibroin fragments used in the silk solution. The finding that the molecular weight of silk fibroin is an important determining factor parameter in controlled production of silk fibroin particles of certain structural features provides a tool to fine-tune silk fibroin particles for certain attributes of interest.

In any of the embodiments contemplated herein, silk fibroin polypeptides of various molecular weights (e.g., fragments) may be used. In some embodiments, for example, provided silk fibroin hydrogel comprises silk fibroin polypeptides having an average molecular weight of between about 3.5 kDa and about 350 kDa. Non-limiting examples of suitable ranges of silk fibroin fragments include, but are not limited to: silk fibroin polypeptides have an average molecular weight of between about 3.5 kDa and about 200 kDa; silk fibroin polypeptides have an average molecular weight of between about 3.5 kDa and about 200 kDa; silk fibroin polypeptides have an average molecular weight of between about 3.5 kDa and about 120 kDa; silk fibroin polypeptides have an average molecular weight of between about 25 kDa and about 200 kDa, and so on. Silk fibroin polypeptides that are “reduced” in size, for instance, smaller than the original or wild type counterpart, may be referred to as “low molecular weight silk fibroin.”

In some embodiments, provided silk fibroin particles are prepared from composition comprising a population of silk fibroin fragments having a range of molecular weights, characterized in that: no more than 15% of total weight of the silk fibroin fragments in the population has a molecular weight exceeding 200 kDa, and at least 50% of the total weight of the silk fibroin fragments in the population has a molecular weight within a specified range, wherein the specified range is between about 3.5 kDa and about 120 kDa.

For more details related to low molecular weight silk fibroins, see: U.S. provisional application concurrently filed herewith, entitled “LOW MOLECULAR WEIGHT SILK FIBROIN AND USES THEREOF,” the entire contents of which are incorporated herein by reference.

While a number of types of silk fibroin may be used to practice the claimed invention, silk fibroin produced by silkworms, such as Bombyx mori, is the most common and represents an earth-friendly, renewable resource. For instance, silk fibroin may be attained by extracting sericin from the cocoons of B. mori. Organic silkworm cocoons are also commercially available. There are many different silks, however, including spider silk (e.g., obtained from Nephila clavipes), transgenic silks, genetically engineered (e.g., recombinant) silk fibroin polypeptides, such as silk fibroin polypeptides from bacteria, yeast, mammalian cells, transgenic animals, or transgenic plants (see, e.g., WO 97/08315; U.S. Pat. No. 5,245,012), and variants thereof, that may be used.

As already noted, an aqueous silk fibroin solution may be prepared using techniques known in the art, with proper adjustment described or referenced herein. Suitable processes for preparing silk fibroin solution are disclosed, for example, in U.S. patent application Ser. No. 11/247,358; WO/2005/012606; and WO/2008/127401. The silk aqueous solution can then be processed into silk matrix such as silk films, conformal coatings or layers, or 3-dimensional scaffolds, or electrospun fibers. A microfiltration step may be used herein. For example, the prepared silk fibroin solution may be processed further by centrifugation and/or filtration, such as syringe based micro-filtration, before further processing. In some embodiments, such step or steps may be carried out to selectively enrich certain subfraction or pool of silk fibroin within a composition.

### A Population of Silk Fibroin Particles

The term “uniform population” refers to a pool of particles (e.g., a composition comprising a plurality of particles), within which one or more parameters that define the structure (i.e., structural features) of such particles are substantially similar. Therefore, a population of particles having a small range or degree of variations with respect to a particular parameter is said to be having a narrow or tight distribution and is characterized as highly uniform. For a number of applications, including biomedical applications, such high uniformity or homogeneity in a particle population is often desirable. By contrast, a population of particles with greater range or degree of variations with respect to a particular parameter is said to be having a wider distribution within the population and is generally less desirable.

A range or degree of variation within a population may be determined by the difference between a maximum value and a minimum value of the parameter(s) observed within the population.

Examples of “parameters” by which the degree of uniformity may be measured include, without limitation, size, density, porosity, material compositions, morphology, such as surface texture, shape, etc. Accordingly, in some embodiments of the invention, a population of silk fibroin particles is uniform in that at least 50% of particles within the population fall within a specified range of one or more of the above-mentioned parameters. In some embodiments, a population of silk fibroin particles is uniform in that no more than 15% of particles within the population fall outside a specified range of one or more of the above-mentioned parameters. In some embodiments, a population of silk fibroin particles is uniform in that with respect to at least one parameter, at least 90%, at least 85%, at least 80%, at least 75%, at least 70%, at least 65% of particles within the population fall within +/−30% of the average value of the parameter observed in the population. For example, in some embodiments, a population of silk fibroin particles is uniform in that with respect to at least one parameter, at least 90%, at least 85%, at least 80%, at least 75%, at least 70%, at least 65% of particles within the population fall within +/−25%, within +/−20%, within +/−15%, or within +/−10% of the average value of the parameter observed in the population.

In some embodiments, a population of silk fibroin particles is uniform in that with respect to at least one parameter, at least 90%, at least 85%, at least 80%, at least 75%, at least 70%, at least 65% of particles within the population fall within +/−30% of the median value of the parameter observed in the population. For example, in some embodiments, a population of silk fibroin particles is uniform in that with respect to at least one parameter, at least 90%, at least 85%, at least 80%, at least 75%, at least 70%, at least 65% of particles within the population fall within +/−25%, within +/−20%, within +/−15%, or within +/−10% of the median value of the parameter observed in the population.

As discussed elsewhere herein, prior art methods for producing silk fibroin particles of a micron-scale took advantage of blending a silk fibroin solution with at least another polymer-based solution, drying the blended materials into a solid form such as a film, mechanically reducing or crushing the solid form into particles, such as by sonication, followed by removing the second polymer material once silk fibroin has been made insoluble. Using such methods, the resulting silk particles typically contain a small amount of residual secondary polymer material, and the particles are less uniform in that there is a greater degree of size and morphological variations and distribution within the final product. Furthermore, because the secondary polymer material is later washed away from the solidified mixture, it leaves behind a “skeleton” or mesh of silk fibroin material, resulting in relatively porous particles.

In some embodiments, for example, a uniform population of silk fibroin particles described herein shows a narrow size distribution such that a majority of particles within the population fall within a specified range of diameters.

In some embodiments, at least 50% of particles within a population have diameters within a specified range, wherein the specified range may be between about 100 nm and 3,000 nm. In some embodiments, at least 55%, at least 60%, at least 65%, at least 70%, at least 75%, at least 80%, at least 85%, at least 90%, at least 95% or greater number of particles within a population have diameters within a specified range. The specified range may be between about 100 nm and about 2,000 nm. In some embodiments, the specified range is between about 100 nm and about 1,800 nm, between about 100 nm and about 1,700 nm, between about 100 nm and about 1,600 nm, between about 100 nm and about 1,500 nm, between about 100 nm and about 1,400 nm, between about 100 nm and about 1,300 nm, between about 100 nm and about 1,200 nm, between about 100 nm and about 1,100 nm, between about 100 nm and about 1,000 nm, between about 100 nm and about 900 nm, between about 100 nm and about 800 nm, between about 100 nm and about 700 nm, between about 100 nm and about 600 nm, between about 100 nm and about 500 nm, between about 100 nm and about 400 nm, between about 100 nm and about 300 nm, between about 100 nm and about 250 nm, or between about 100 nm and about 200 nm. In some embodiments, the specified range may be between about 500 nm and about 3,000 nm, e.g., between about 500 nm and about 3,000 nm, between about 600 nm and about 3,000 nm, between about 700 nm and about 3,000 nm, between about 800 nm and about 3,000 nm, between about 900 nm and about 3,000 nm, between about 1,000 nm and about 3,000 nm, between about 1,100 nm and about 3,000 nm, between about 1,200 nm and about 3,000 nm, between about 1,300 nm and about 3,000 nm, between about 1,400 nm and about 3,000 nm, between about 1,500 nm and about 3,000 nm, between about 1,600 nm and about 3,000 nm, between about 1,700 nm and about 3,000 nm, between about 1,800 nm and about 3,000 nm, between about 1,900 nm and about 3,000 nm, between about 2,000 nm and about 3,000 nm, between about 2,100 nm and about 3,000 nm, between about 2,200 nm and about 3,000 nm, between about 2,300 nm and about 3,000 nm, between about 2,400 nm and about 3,000 nm, between about 2,500 nm and about 3,000 nm, between about 2,600 nm and about 3,000 nm, between about 2,700 nm and about 3,000 nm, between about 2,800 nm and about 3,000 nm, or between about 2,900 nm and about 3,000 nm.

In some embodiments, a uniform population of silk fibroin particles is uniform in that less than a certain percentage of particles within the population fall outside a specified diameters. For example, in some embodiments, less than a certain percentage of the number of particles within a population has diameters exceeding a specified diameter. In some embodiments, less than a certain percentage of the number of particles within a population has diameters below a specified diameter.

### Co-Flow Method

In a broad sense, the invention provides means for achieving controlled fluid interactions. Typically, it is achieved by providing a device that allows controlled deposition of “droplets” from a discrete phase (such as a silk fibroin solution) into a continuous phase, wherein the two phases are immiscible with each other. In some embodiments, each of such droplets is in motion (i.e., flowing) with respect to its surrounding immiscible solution in the continuous phase. In some embodiments, the immiscible solution in the continuous phase is flowing at a certain flow rate. In some embodiments, the solution in the discrete phase may also be flowing at a certain flow rate, although not required.

Thus, provided herein is a simple and effective approach for the synthesis of silk fibroin micro- and submicron particles (e.g., spheres) using a co-flow method. In some embodiments, a continuous phase of an immiscible solution (such as PVA) comes in direct contact with (e.g., by being flowed over) a discrete phase of a silk solution. This process allows the generation of uniform silk particles without filtering, with simple purification steps, and with the possibility to generate consistently sized micro- and submicron spheres with tunable diameters. Sphere diameter may be controlled by varying parameters, such as the concentration of the immiscible solution of for instance PVA, silk concentration, the flow rate ratios, silk molecular weight distribution, or any combination of these parameters.

Techniques described herein allow control over the silk fibroin particle size on a per particle basis, which allows for a greater degree of control over the production of a composition of uniformly sized particles. Such a process of creating silk particles identically may also provide control over other physical and structural properties of the silk particle in addition to size such as porosity, morphology, surface roughness, hardness, specific weight, fracture toughness, shear strength, ductility, etc. Such a process may also provide control over optical characteristics of the silk particles such as color, absorptivity, luminosity, reflectivity, refractive index, scattering, and transmittance and electromagnetic characteristics of the silk particles such as electrical conductivity, dielectric constant, permittivity, piezoelectric constants, diamagnetism, hysteresis, and permeability.

Techniques described herein are capable of producing uniform compositions of silk fibroin particles that have similar parameters on a per particle basis. Since each silk particle is generated from the same discrete phase solution by being subject to identical immiscible solution or solutions, the resulting silk particles will exhibit similar values of certain parameters to form a uniform composition. These parameters by which the degree of uniformity may be measured include, without limitation, size, density, porosity, material compositions, morphology, such as surface texture, shape, hardness, specific weight, fracture toughness, shear strength, ductility, color, absorptivity, luminosity, reflectivity, refractive index, scattering, transmittance, electrical conductivity, dielectric constant, permittivity, piezoelectric constants, diamagnetism, hysteresis, and permeability, etc.

Without wishing to be bound by a particular theory, droplet formation occurs by break-off of the discrete phase in the bulk fluid stream leading to the formation of monodispersed droplets. Self-assembly of silk fibroin in the droplets produces consistently sized spheres in the micron or submicron range. The type of break-off of the droplet is determined by the volumetric flow rates of the continuous and discrete flows of the two immiscible fluids, the interfacial tension, and viscosity of the fluids. This in turn determines the size of the droplets that break-off from the initial stream.

In some embodiments, a silk fibroin solution having a specific parameter value and an immiscible solution having a particular parameter value are provided to produce a silk fibroin sphere. The parameter values of the silk fibroin solution and immiscible solution that are controlled are viscosity, flow rate, molecular weight, solution boiling duration, and concentration. Controlling at least one of these parameter values for either the immiscible solution or the silk fibroin solution allows for the production of a silk sphere with a controllable diameter. The silk fibroin solution is introduced into the immiscible solution to produce a monodispersed droplet (i.e, droplet having particles of a uniform size in a dispersed phase). The introduction of the silk fibroin solution into the immiscible solution results in a net movement between the silk fibroin solution and the immiscible solution which induces a force on the droplet.

Upon condensation, the monodispersed droplet will result in a silk fibroin sphere having a diameter within a specified controllable range. The diameter value can be controlled by modifying at least one of viscosity, flow rate, molecular weight, solution boiling duration, and concentration of either the either the immiscible solution or the silk fibroin solution. Modifying one of these parameter values affects the droplet size (i.e., droplet diameter, droplet volume, etc.) of the monodispersed droplet, which in turn affects the size of the resulting silk particle that condenses from the droplet.

A force induced on the droplet, resulting from the net movement between the silk fibroin solution and the immiscible solution, affects droplet size and thereby affects the diameter of the silk fibroin sphere resulting from the condensation of the droplet. The force induced on the droplet may affect how much silk fibroin solution is dispensed per droplet, thereby affecting droplet size.

In some embodiments, after the silk fibroin solution droplet is pinched off from the rest of the silk fibroin solution, the silk fibroin in the monodispersed droplet self assembles during condensation of the silk fibroin solution to form a silk fibroin sphere. The amount of silk fibroin within a monodispersed droplet is directly proportional to the diameter of the resulting silk sphere. Accordingly, by controlling at least one of viscosity, flow rate, molecular weight, solution boiling duration, and concentration values for either the immiscible solution or the silk fibroin solution allows for the production of a silk sphere with a controllable diameter in a specified range. In another embodiment, the silk fibroin may condense to form a particle of a non-spherical shape as well where the techniques described herein affect the particle size of the silk particle.

In some embodiments, the monodispersed droplet is deposited on a substrate to form a condensed silk sphere after it has been introduced into the immiscible solution. Any immiscible solution in contact with the condensed silk sphere is removed by centrifuging the condensed silk sphere with ultrapure water.

While such a technique allows for sequential droplet formation, droplet formation may also occur in parallel, in some embodiments, by using multiple containers of identical silk fibroin solution feeding into the same immiscible solution.

In some embodiments, a uniform silk sphere composition is produced. The uniform silk composition includes multiple silk fibroin spheres having diameters within a specified range. To produce such a uniform silk sphere composition, a silk fibroin solution having a specific parameter value and an immiscible solution having a particular parameter value are provided. The parameter values of the silk fibroin solution and immiscible solution that are controlled are viscosity, flow rate, molecular weight, solution boiling duration, and concentration. Controlling at least one of these parameter values for either the immiscible solution or the silk fibroin solution allows for the production of multiple silk spheres with controllable diameters. The silk fibroin solution is introduced into the immiscible solution to produce multiple monodispersed droplets. The introduction of the silk fibroin solution into the immiscible solution results in a net movement between the silk fibroin solution and the immiscible solution which induces a force on each of the droplets. The induced force affects droplet size of each of the droplets and thereby affects the diameter of the silk fibroin spheres resulting from the condensation of the droplets. The force induced on the droplets may affect how much silk fibroin solution is dispensed per droplet, thereby affecting droplet size.

In some embodiments, the force induced on the silk droplets by the net movement between the silk fibroin solution and the immiscible solution is imparted onto the silk fibroin solution to pinch off portions of the silk fibroin solution to produce multiple monodispersed droplets.

In some embodiments, each monodispersed droplet created is subject to the same net movement and the same force as other monodispersed droplets used to produce the uniform silk fibroin sphere composition.

Typically, techniques described herein are capable of generating silk fibroin droplets of volumes as small as in a ˜picoliter (pL) range. Upon condensation, such droplets can then form silk fibroin particles (e.g., spheres) having diameters in micron- to submicron (i.e., nano) ranges, depending on additional parameters discussed herein.

Of particular relevance in the generation of silk fibroin droplets and subsequent formation of silk fibroin-based submicron particles is likely the jetting regime. Devices operating in the jetting regime have been shown to generate smaller diameter particles compared to the dimensions of the outlet orifice, in agreement with what is observed here. In this case, both fluids are aqueous solutions with low interfacial tension that leads to a reduction in the driving force for liquid jets to break-off into droplets.

Once the droplet of the discrete phase is formed silk will condense because its immiscibility with PVA, following the same mechanism reported previously. With respect to the previous work on the silk spheres formation in a PVA solution bath the possibility to condense the sphere from a droplet of defined and consistent size allows the possibility to produce silk micron- and submicron spheres with better control on the size distribution. Since the droplet diameter can be controlled by changing fluid parameters like flow rate and viscosity, particles (e.g., spheres with) controlled and tunable size can be synthesized.

### Discrete Phase

The term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water.

The silk fibroin (silk fibroin protein, i.e., silk fibroin peptides or fragments thereof) can be present in the solution at any concentration suited to the need. In some embodiments, one of the limiting factors may be a threshold concentration beyond which a silk fibroin solution may solidify (e.g., gellation, etc.) due to shear force or stress imposed on the solution. For example, in some embodiments, silk fibroin solution used as the discrete phase of the co-flow method may flow through and/or deposited through a channel or tubular structure, such as a needle. In such situations, the flowability (may be referred to as injectability in the context of a needle) of the silk fibroin solution is affected by a number of factors, such as the concentration of the silk fibroin in the solution, its molecular weight range (e.g., average fragment size), the flow rate or force being exerted on or within the discrete phase, the viscosity of the solution, the dimensions (e.g., diameter, cross section area, length, etc.) of the channel or tubular form through which the solution is to flow. These parameters may determine or affect the amount of shear stress (force) created, which in turn may trigger gellation or self-assembly upon reaching a certain threshold.

In some embodiments, the aqueous silk fibroin solution can have silk fibroin at a concentration of about 0.1% wt/v to about 90% wt/v, 0.1% wt/v to about 75% wt/v, or 0.1% wt/v to about 50% wt/v. In some embodiments, the aqueous silk fibroin solution can have silk fibroin at a concentration of about 0.1% wt/v to about 10% wt/v, about 0.1% wt/v to about 5% wt/v, about 0.1% wt/v to about 2% wt/v, or about 0.1% wt/v to about 1% wt/v. In some embodiments, the silk fibroin solution have silk fibroin at a concentration of about 10% wt/v to about 50% wt/v, about 20% wt/v to about 50% wt/v, about 25% wt/v to about 50% wt/v, or about 30% wt/v to about 50% wt/v.

In some embodiments, silk fibroin solutions may be prepared by reconstitution of a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions. To give but one example, silk fibroin microspheres or particles suspended in a solution do not themselves constitute a silk fibroin solution.

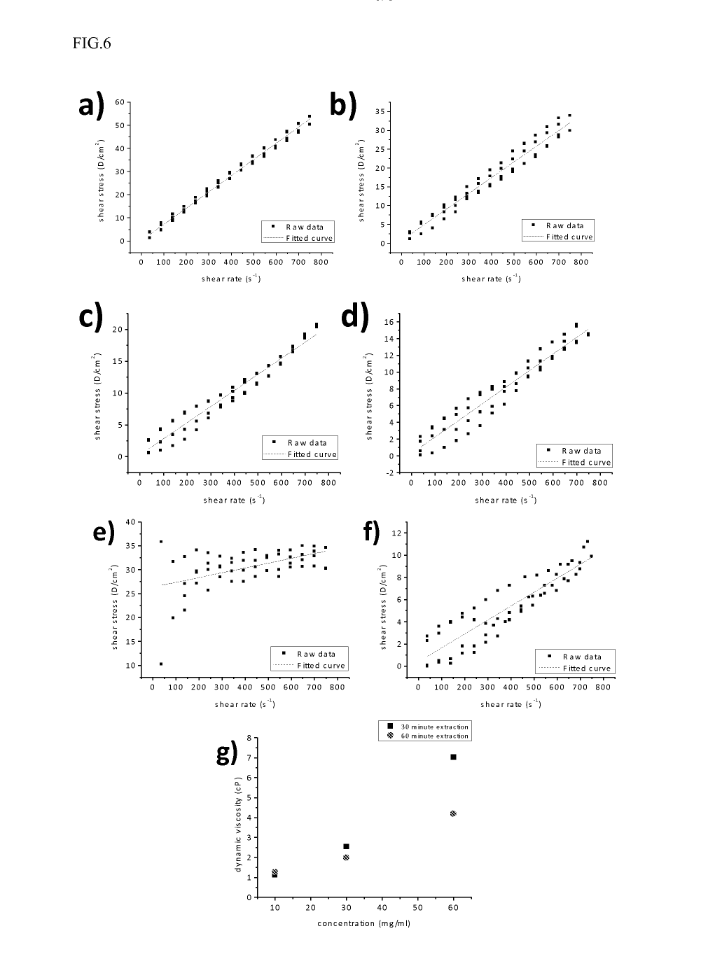

In the case of silk solution an increase in either the molecular weight of the silk solution or in its concentration results in an increase of its viscosity. The viscosity affects the break-off condition (k* term in the equation), and a larger discrete phase viscosity will generate smaller particles. Control of the silk fibroin molecular weight can be achieved during silk solution preparation, for example, by using different boil times or heating conditions when processing the native silk fibers. Different silk concentrations can be obtained by dilution of the solution with water. For 30 minute-boil silk, the measured viscosities are 1.12 cP, 2.53 cP, and 7.01 cP for 10 mg/ml, 30 mg/ml, and 60 mg/ml samples respectively, whereas for 60 minute-boil silk, the viscosities are 1.25 cP, 1.98 cP, and 4.18 cP for 10 mg/ml, 30 mg/ml, and 60 mg/ml samples respectively as measured by a Brookfield viscometer. The viscosity was calculated using the Bingham model for viscoplastic materials defined as τ=τ₀+ηD where τ is the shear stress, τ₀ is the yield stress, D is the shear rate, and η is the viscosity. Solving for the slope of the graph plotted with shear stress vs. shear rate provides the measured viscosity (FIG. 6).

### Characterization of Solutions

Physical properties of solutions, such as silk fibroin solutions (e.g., solutions comprising a dissolved silk fibroin polypeptide, optionally further comprising an active agent and any excipient) can also be evaluated in order to characterize the solutions. Common parameters measured to characterize such properties may include, without limitation: solution density/specific gravity, viscosity, surface tension, refractive index, turbidity, osmolarity, boiling and/or melting points, and any combination thereof. The art is familiar with suitable techniques that can be employed to measure any of the properties.

Density is simply the measurement of mass per unity of volume. Dimensionless values of density (e.g., specific gravity or relative density) can also be evaluated, by creating a ratio of the density of the silk fibroin solutions to the density of water, under the same conditions. Density of a solution can vary based on a variety of factors, including, but not limited to temperature, pressure and concentration of solutes. In particular, the density of the silk fibroin solution can increase or decrease depending on the concentration of silk fibroin, concentration of various excipients, as well as the temperature and pressure, as compared to water at the same conditions. In some embodiments, the density of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%.

Density, specific gravity (i.e., relative density) can be measured in a variety of techniques, including, but not limited to the use of a pychnometer, hydrostatic pressure based instruments, vibrating element or ultrasonic transducers, radiation based devices and buoyancy measurements.

Viscosity of the various silk fibroin solutions can also be measured to characterize the solution. Viscosity is the measure of a fluid's resistance to being deformed (e.g., by shear or tensile stress), commonly considered a fluid's resistance to flow. Fluids can be characterized by how their viscosity changes in response to a condition change, e.g., Newtonian fluids have a constant viscosity over a range of changing stress, while non-Newtonian fluids have a changing viscosity over a range of changing stresses. Viscosities of solutions, e.g., silk fibroin solutions can also vary greatly over a range of temperature and pressure conditions. In some embodiments, the viscosity of the silk fibroin solution can increase or decrease depending on the concentration of silk fibroin, concentration of various excipients, as well as the temperature and pressure, as compared to water at the same conditions. In some embodiments, the viscosity of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%

Viscosity can be measured in a variety of techniques, including, but not limited to the use of a Brookfield viscometer, Ostwald viscometer (i.e., U-tube viscometers), piston based viscometers, oscillating or vibrating viscometers, and rotational viscometers.

Surface tension of various silk fibroin solutions can also be measured and used to characterize the solution. Surface tension is the contractive force on the surface of a liquid that to resist and external force. It results from the fact that molecules on the surface of a fluid do not have forces acting on them equally from all directions, and are therefore pulled in, causing contraction of the surface. In particular, the surface tension of the silk fibroin solution can increase or decrease depending on the concentration of silk fibroin, concentration of various excipients, as well as the temperature and pressure, as compared to water at the same conditions. In some embodiments, the surface tension of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%.

Surface tension can be measured by a variety of techniques and instruments. Exemplary methods include, but are not limited to, the Du Noüy ring method, the Du Noüy-Paddy method, spinning and pendent drop methods, Wilhelmy plate methods, among others. Refractive index is another property of various silk fibroin solutions that can be used to characterize and identify a solution. Refractive index is the degree that light is bent as it passes from one medium to another (e.g., from water to air, or from a solution to air). The refractive index of a solution can be used to determine a variety of analytical values, including but not limited to concentration and composition. Additionally, refractive index can be used in calibrating other analytical measurements that rely on light or electromagnetic radiation (e.g., Doppler based particle analysis systems, IR systems etc.).

In particular, the refractive index of the silk fibroin solution can increase or decrease depending on the concentration of silk fibroin, concentration of various excipients, as well as the temperature and pressure, as compared to water at the same conditions. In some embodiments, the refractive index of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%. Any conventional refractometer can be used to evaluate a silk fibroin solution's refractive index.

In some embodiments, turbidity of the silk fibroin solutions can be measured to characterize the solution. While generally, the silk fibroin solutions do not exhibit any cloudiness, or suspended particles (e.g., the silk fibroin solutions are “water white”) turbidity can be used in quality control operations. In particular, the turbidity of the silk fibroin solution can increase or decrease depending on the concentration of silk fibroin, concentration of various excipients, as well as the temperature and pressure, as compared to water at the same conditions. In some embodiments, the turbidity of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%. Turbidity can be measured by any conventional turbidimeter.

In some embodiments, the osmolarity of the solution can be measured as a way to characterize and evaluate silk fibroin solutions. Osmolarity refers to the solute concentration in a solution, generally for compounds that disassociate. Similarly, the tonicity of silk fibroin solutions in comparison to another fluid (e.g., water) can be used to evaluate solutions. In particular, the osmolarity of the silk fibroin solution can increase or decrease depending on the concentration of silk fibroin, concentration of various excipients, as well as the temperature and pressure, as compared to water at the same conditions. In some embodiments, the osmolarity of the silk fibroin solution changes (e.g., increases or decreases) by about 0.1-25%, by about 0.1-5%, by about 1-10%, by about 10-25%, e.g., 0.1%, 0.5%, 1%, 2%, 3%, 4%, 5%, 6%, 7%, 8%, 9%, 10%, 11%, 12%, 13%, 14%, 15%, 16%, 17%, 18%, 20%, 21%, 22%, 23%, 24% and 25%.

In some embodiments, techniques described herein may be combined with one or more other technologies for the analysis and characterization of silk fibroin solutions. For example, in some embodiments, silk fibroin solutions are characterized using one or more of chromatographic methods, electrophoretic methods, nuclear magnetic resonance methods, and combinations thereof. Exemplary such methods include, for example, NMR, mass spectrometry, liquid chromatography, 2-dimensional chromatography, SDS-PAGE, antibody staining, lectin staining, monosaccharide quantitation, capillary electrophoresis, micellar electrokinetic chromatography (MEKC), and combinations thereof.

In some embodiments, silk fibroin solutions can be analyzed by chromatographic methods, including but not limited to, liquid chromatography (LC), high performance liquid chromatography (HPLC), ultra performance liquid chromatography (UPLC), thin layer chromatography (TLC), amide column chromatography, and combinations thereof.

In some embodiments, silk fibroin solutions can be analyzed by spectroscopic methods, including but not limited to infrared spectroscopy (IR) and related variations, Fourier transform spectroscopy and related variations, Raman spectroscopy and related variations, circular dichroism (CD) and related variations, linear dichroism, magnetic circular dichroism, force spectroscopy, and combinations thereof.

In some embodiments, silk fibroin solutions can be analyzed by mass spectrometry (MS) and related methods, including but not limited to, tandem MS, LC-MS, LC-MS/MS, matrix assisted laser desorption ionization mass spectrometry (MALDI-MS), Fourier transform mass spectrometry (FTMS), ion mobility separation with mass spectrometry (IMS-MS), electron transfer dissociation (ETD-MS), time of flight mass spectrometry, electrospray ionization and combinations thereof.

In some embodiments, silk fibroin solutions can be analyzed by electrophoretic methods, including but not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific silk fibroin solution components, and combinations thereof.

In some embodiments, silk fibroin solutions can be analyzed by nuclear magnetic resonance (NMR) and related methods, including but not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

### Continuous Phase

As already discussed, in a co-flow system, a “continuous phase” refers to a body of an immiscible solution, into which one or more droplets of a solution from a “discrete phase” are deposited. In a context of the present disclosure, the phrase “an immiscible solution” is used to refer to a solution of the continuous phase with which silk fibroin-based droplets come in direct contact and which does not readily mix with the silk fibroin solution.

According to the invention, an immiscible solution is provided typically in a container, into which one or more droplets of a silk fibroin solution are deposited, thereby coming directly in contact with the external surface of each of such silk fibroin droplets. As discussed in more detail herein, the interaction between the silk fibroin droplets and the immiscible solution surrounding such droplets facilitates condensation of silk fibroin, resulting in the formation of silk fibroin spheres.

At the molecular level, the interactions likely involve transfer of water molecules from the silk fibroin solution (droplets) to the surrounding immiscible solution in the process of the silk fibroin particle formation. The result is the formation of silk fibroin particles that are substantially spherical in shape, having a smooth surface morphology with relatively low porosity, and are substantially free of contamination with other materials. It should be noted that there is no material mixing or blending of the silk fibroin solution (i.e., droplets) and the surrounding immiscible solution in the process, such that the silk fibroin particles (e.g., spheres) themselves do not contain the immiscible solution, which can be readily washed away.

Although the skilled artisan will readily understand that different immiscible solutions may require different concentrations to be useful, typically, useful concentrations of an immiscible solution for carrying out the invention described herein range between about 0.5 vol % and 20 vol %, e.g., about 0.5%, about 1.0%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%. For instance, in the case of PVA, concentrations between about 2% and about 10% are suitable, e.g., about 2%, about 3%, about 4%, about 5%, about 6%, about 7%, about 8%, about 9%, or about 10% of PVA dissolved in water.

According to the invention, a certain volume of a silk fibroin solution is released into an immiscible solution from an opening as individual droplets. At the time and site of release, each of the silk fibroin droplets is in motion with respect to the surrounding immiscible solution of the continuous phase. In some embodiments, the surrounding immiscible solution is in motion such that it is flowing in a container (e.g., a chamber) which may or may not be a closed container.

In some embodiments, the silk fibroin solution is dispensed from a container though a small opening in the container into an immiscible solution flowing around the container. The silk fibroin solution may be in contact with the immiscible solution flowing around the container. The immiscible solution flowing along the container containing the silk fibroin solution may affect the amount of silk fibroin dispensed from the container in one droplet of the silk fibroin solution based on the interaction between the silk fibroin and the immiscible solution when the immiscible solution flows past the opening of the container (i.e., release site) dispensing the silk fibroin solution. At the opening of the container dispensing the silk fibroin solution, the immiscible solution flowing past the growing droplet of silk fibroin solution may pinch off the silk fibroin solution. The droplet dispensed from the silk fibroin solution dispensing container may contain immiscible solution mixed with the silk fibroin solution.

Preferably, the immiscible solution in the container is in motion at a certain flow rate. In some embodiments, the immiscible solution in the container is flowing at a predetermined flow rate. In some embodiments, the immiscible solution is flowing at different predetermined flow rates at different portions of its container due to the shape and structure of the container. For example, the container may include multiple channels, or microchannels, capillaries, sharp microchannel turns, and additional tubular structures that may impede the flow of the immiscible solution, thereby changing the flow rate of the immiscible solution once the solution encounters these structures. Additionally, the container may include portions that are not flat surfaces and include ramps, curves that may modify the flow rate of the immiscible solution once the immiscible solution encounters such surfaces. In some embodiments, the immiscible solution is flowing at different flow rates due to interactions with additional fluids or gases present in the container containing the immiscible fluid. The interaction between the immiscible solution and these additional fluids or gases present in the container may apply a net force on the immiscible solution, thereby causing the flow rate of the immiscible rate to increase or decrease. In some embodiments, the immiscible solution in the container is flowing at a constant flow rate. In some embodiments, the flow of the immiscible solution in the container is essentially unidirectional.

Such essentially unidirectional flow of an immiscible solution may be created, for example, by flowing the immiscible solution through an elongated body of container, such as channels, including microchannels, and tubular structures (i.e., tubes), such as needles and capillaries. In some embodiments, such elongated body of containers through which an immiscible solution flows may be a flexible or rigid structure. For example, in some embodiments, tubular structures useful for carrying out the present invention include needles and channels.

In some embodiments, the release site of silk fibroin droplets itself may be in motion with respect to the surrounding immiscible solution. For example, the silk fibroin droplets may be discharged from a container into the immiscible solution. The release site of the silk fibroin droplets may be an opening of such a container that is surrounded by the immiscible solution. In some embodiments, the container discharging the silk fibroin droplets may be in motion with respect to the immiscible solution that is not flowing. The opening of the container may be moving with respect to the static immiscible solution. The silk fibroin droplets at the release site will be subject to forces resulting from the movement of the silk droplets being discharged with respect to the surrounding static immiscible solution. In some embodiments, the container discharging the silk fibroin droplets may be fixed in position while the immiscible solution is flowing around the opening of the container from which the silk fibroin droplets are being discharged. The immiscible solution moving around the silk fibroin droplets at the release site is in contact with the silk fibroin droplets being formed. The silk fibroin droplets at the release site will be subject to forces resulting from the movement of the immiscible solution around the droplets. In some embodiments, both the container dispensing the silk fibroin droplets and the immiscible solution are moving. The net movement of the silk fibroin droplets against the immiscible solution flowing around the release site of the container induces a force on the silk fibroin droplets.

In the context of the present disclosure, a suitable immiscible solution contains at least one base substance (i.e., a solute) having a preferred range of molecular weight and/or concentration that makes up the immiscible solution.

Additionally or alternatively, in some embodiments, a suitable immiscible solution in accordance with the present invention has a preferred range of viscosity, as exemplified in the Examples below.

In the context of the present disclosure, suitable immiscible solutions are typically water-soluble polymer solutions. For example, suitable immiscible solutions include, but are not limited to, water-soluble polyesters and polymer alcohols. In some embodiments, suitable polyesters include aliphatic polyesters, semi-aromatic polyesters, and aromatic polyesters. In any of such embodiments, polyesters may be a homopolymer (i.e., repeating units of a single monomer) or a copolymer (i.e., repeating units including at least two types of monomers).

In some embodiments, suitable polyesters useful for carrying out the present invention include, but are not limited to: Polyglycolide or Polyglycolic acid (PGA); Polylactic acid (PLA); Polycaprolactone (PCL); Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate) (PHBV); Polyethylene terephthalate (PET); Polybutylene terephthalate (PBT); Polytrimethylene terephthalate (PTT); Polyethylene naphthalate (PEN); Poly(lactic-co-glycolic acid) (PLGA); Vectran; and any combination thereof.

In some embodiments, suitable polymer alcohols include hydroxylated polymers (also referred to as polyhydroxylated polymers or polyhydric polymers), such as Polyvinyl alcohol (PVA), which is a synthetic polymer derived from polyvinyl acetate through partial or full hydroxylation. Generally, polymer solutions useful for carrying out the present invention have low protein adsorption characteristics, biocompatibility, high water solubility, and chemical resistance.

### Means of Controlling Fluid Dynamics (Fluid-Fluid Interactions)

In a broad sense, a device that allows production of silk fibroin particles by facilitating controlled deposition of droplets from a discrete phase (such as a silk fibroin solution) into a continuous phase as described herein. The device allows for a simple and effective synthesis of silk fibroin micro- and submicron particles (e.g., spheres) using the co-flow method described throughout this disclosure. The device allows a discrete phase solution to come into contact with (e.g., by being flowed over, by deposition) a continuous phase solution (e.g., immiscible solution). The device allows the continuous phase solution to exert forces (e.g., drag force, capillary force, buoyant force, viscous force, etc.) on the discrete phase solution in order to control the deposition rate of discrete phase droplets into the continuous phase solution.

In some embodiments, the co-flow device includes a body on which the continuous phase solution rests. Such a body may comprise, without limitation, a planar or a curved surface, a closed container, a channel, or any combination thereof, in which the continuous phase solution is either flowing or rests. In some embodiments, such a body provides a medium on which the continuous phase solution may be flowing at a constant rate. The flow of the continuous phase solution on such a body may be affected by the structure, orientation, and size of the body. For instance, the continuous phase solution may flow in a network of channels, some that mix and intermingle with one another. The body in which the continuous phase solution may be flowing in may contain sharp turns and obstructions that reduce the flow rate of the device. Different portions or the entire body on which the channel may be flowing may be tilted at an angle that allows gravitational forces to accelerate or decelerate the flow rate of the continuous phase solution. Additionally, the body on which the continuous phase solution may be flowing on may contain narrow channels which subject any fluids flowing within them to capillary forces, thereby affecting the flow rate of such fluids.

In some embodiments, the continuous phase solution may be resting statically on the body. The body may be a flat surface that allows the continuous phase to sit undisturbed until it is subjected to the discrete phase solution.

In some embodiments, the body containing the continuous phase solution may be in contact with a container dispensing the discrete phase solution (e.g., silk fibroin solution). The container is not be limited to any particular shape or structure, but contains an opening through which its fluidic contents (i.e., solution of the discrete phase) may be dispensed. The body, on which the continuous phase solution is located, is positioned with respect to the container such that the opening of the container has direct access to the continuous phase solution. The opening of the container may be directly in contact with a surface of the body containing the continuous phase solution. In another embodiment, the opening of the container may be positioned such that it is directly above the surface on which the continuous phase solution rests or flows, such that when the discrete phase droplets are dispensed from their container, the droplets fall on (and/or at least in part “pulled by”) the continuous phase solution. In another embodiment, the container may be partially or fully encapsulated by the body containing the continuous phase solution. The continuous phase solution may flow around the discrete phase solution container, all the while exerting pressure on the container, and the discrete phase solution located within the container, that affects the rate of discrete phase solution droplet formation. In some embodiments, the opening of the discrete phase solution container is completely surrounded by the continuous phase solution. In some embodiments, the container is at least partially positioned inside the body such that the continuous phase solution surrounds the opening of the container. The discrete phase solution droplets forming at the opening of such a container may be subject to forces exerted by any interaction between it and the continuous phase solution surrounding it. These forces may affect the droplet formation rate.

At the release site of the discrete phase solution (e.g., the opening of the container), where each droplet forms, there is a net movement between the discrete phase solution and the continuous phase solution. In some embodiments, the release site of silk fibroin droplets itself may be in motion with respect to the surrounding immiscible solution. For example, the container discharging the silk fibroin droplets may be in motion with respect to the static continuous solution resting on the surface of the body containing it. The co-flow device may contain a mechanism (e.g., robot arm, spring loaded cantilever set into motion by an electrical signal, etc.) that sets the container, or at least the opening of the container, containing the discrete phase solution into motion with respect to the static continuous phase solution. The opening of the container moves against the static continuous phase solution, subjecting the discrete phase solution at the opening of the container to forces that affect the rate of droplet formation. In some embodiments, the container discharging the silk fibroin droplets may be fixed in position while the immiscible solution is flowing around the opening of the container from which the silk fibroin droplets are being discharged. In some embodiments, both the container dispensing the silk fibroin droplets and the immiscible solution are moving.

The net movement of the silk fibroin droplets against the immiscible solution flowing around the release site of the container induces a force or several forces (e.g., capillary forces, drag forces, etc.) on the silk fibroin droplets. The net force resulting from these individual forces exerted on the discrete phase solution inside its container causes the discrete phase solution to form droplets, which are dispensed into the continuous phase solution. In other words, the magnitude of the induced force on the discrete phase solution affects the portion of dispersed phase solution discharged in a droplet from the container into the body. One or more of the properties of the discrete phase solution, such as viscosity, flow rate, molecular weight, solution boiling duration of the discrete phase solution, and concentration of the discrete phase in the discrete phase solution affect how the discrete phase solution reacts to the forces subjected to it during droplet formation. In addition, the viscosity, flow rate, molecular weight, solution boiling duration of the continuous phase solution, and concentration of the continuous phase solution also affect the interaction between the continuous phase solution and the discrete phase solution and affect the magnitude of the forces that the discrete phase solution is subjected to the release site of the discrete phase solution. These factors affect the amount of discrete phase solution that is dispensed in a droplet, which in turn will affect the size and/or other parameters of silk fibroin particles.

In some embodiments, the resulting discrete phase solution droplets enter a region of the co-flow device that includes a mixture of the continuous phase solution and the dispersed phase solution discharged from the opening of the container. In some embodiments in which the continuous phase solution is previously flowing, the droplets may flow with the continuous phase solution. In other embodiments, at which the continuous phase solution is not flowing, the droplets introduced into the continuous phase solution may cause the continuous phase solution to flow. In yet another embodiment, the droplets dispensed into the continuous phases solution may be static upon their introduction into the continuous phase solution.

In some embodiments, the region of the co-flow device containing such a mixture has direct access to a collection vessel on which the mixture is deposited. The collection vessel may be a substrate on which the mixture is deposited from the co-flow device. In some embodiments, the collection vessel may be connected to the region of the co-flow device containing the mixture of discrete phase droplets and continuous phase solution. For instance, the collection vessel may be a channel, a tube, or a similar apparatus that collects the mixture of the discrete phase droplets and continuous phase solution from the co-flow device for deposition onto a substrate on which the mixture may rest in a static condition. In some embodiments, the mixture is transferred onto the collection vessel on which the dispersed phase solution condenses to form silk particles. In some embodiments, collection vessel is or comprises a filter, onto which silk fibroin particles may be collected.

Condensation of the discrete phase droplets at least in part affects the silk particle size and characteristics. In some embodiments, such condensation occurs when the liquid phase in the discrete phase solution is at least in part separated from the silk fibroin in the discrete phase solution. In some embodiments, the silk fibroin self assembles during droplet condensation. This condensation step may occur in the region of the continuous phase solution container containing the mixture of the discrete phase droplets and the continuous phase solution, in the collection vessel, or any combination thereof.

The mixture of droplets, silk fibroin particles, and continuous phase solution collected collection vessel is dried (e.g., heated in an oven chamber, dried in ambient conditions, etc.) to allow droplet condensation to complete. In some embodiments, silk fibroin particles may optionally be crosslinked by any suitable method known in the art, e.g., exposure to ultraviolet radiation. Any continuous phase solution remaining (whether in liquid or solid form) is removed from the dried silk particles by centrifuging the dried silk fibroin particles in ultrapure water.

### Co-Flow Device.

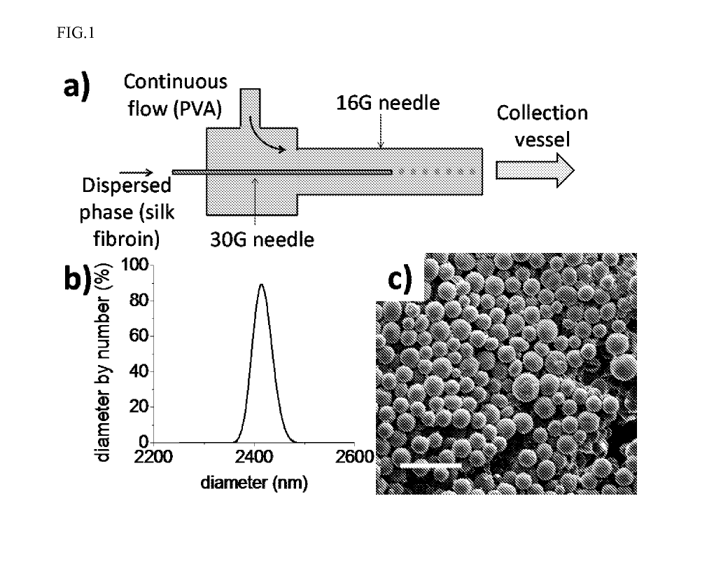

In some non-limiting embodiments, as shown in FIG. 1A, the co-flow device is a coaxial device composed of two elongated hypodermic needles that form a concentric coaxial device. The body containing the continuous phase solution is a large hypodermic needle with a smaller needle gauge value (and therefore a wider needle width) than the smaller hypodermic encapsulated at least partially within it. The elongated smaller hypodermic needle contains the discrete phase solution. An elongated outer channel is formed in the space between the needle tube of the larger hypodermic needle and the wall of the smaller hypodermic needle located within the larger needle. The continuous phase solution flows in this outer channel, often exerting forces against the discrete phase solution contained in the smaller hypodermic needle that the continuous phase solution is flowing along. The two hypodermic needles are positioned in a manner such that an end of the smaller hypodermic needle, which dispenses the discrete phase solution as droplets into the continuous phase solution in the outer channel, is located within the larger hypodermic needle. The region between the end of the smaller hypodermic needle (e.g., the release site of the discrete phase solution) and the end of the larger hypodermic needle contains a mixture of the discrete phase droplets that are dispensed from the smaller hypodermic needle into the larger hypodermic needle. The mixture may flow in this region until it is dispensed from the larger hypodermic needle into a collection vessel. In some embodiments, the silk fibroin in the discrete phase solution droplet may condense into a silk fibroin particle by self-assembly in this region.

In some embodiments, the hypodermic needles used to form the coaxial co-flow device may be attached to hypodermic needle plungers that can be pressed by a user or a mechanical device to expel the contents of the hypodermic needles. As a result, the continuous solution and discrete solution may be subjected to additional pressure that affects their flow rates by such a hypodermic needle plunger expelling the contents of the hypodermic needles. Such additional pressure may be variable and may affect certain aspects of the formation of silk fibroin particles. For example, it may affect the rate of droplet formation and condensation of the silk fibroin in the discharged discrete phase solution droplets flowing in the region of the outer channel containing a mixture of the discrete phase solution droplets and the continuous phase solution.

In some embodiments, the co-flow device is created using non-tubular channels. For instance, the larger outer channel containing the continuous phase solution and the small inner channel contained at least partially within the larger outer channel filled with the discrete phase solution may be microfluidic channels. These microfluidic channels may contain micropumping systems and valves throughout the channels in order to maintain a controllable pressure and flow rate of the fluids flowing through them.

In some embodiments, not depicted in FIG. 1A, the inner and outer channels used to form the co-flow device may include additional hydrodynamic trapping structures (i.e., tubular structures, beads, cells, etc.) that impede the flow rate of the continuous and discrete phase solutions flowing in these hypodermic needle channels. The channel width of the inner and outer channel may be designed to induce capillary forces on the fluids flowing within them to control flow rate.

In some embodiments, the co-flow channels containing the discrete phase solution and the continuous phase solution may be constructed of a semipermeable membrane. Such semipermeable membranes may be designed to be contain the discrete and continuous phase solution but to allow smaller impurities to be filtered out.

In some embodiments, no portion of the channel containing the discrete phase solution is encapsulated or positioned within the channel containing the continuous phase solution. An end of the discrete phase solution channel may be in contact with a portion of the channel containing the continuous phase solution. In an implementation, the channel containing the discrete phase solution and the channel containing the continuous phase solution is arranged as a T-shaped structure. In an implementation, the channel containing the discrete phase solution and the channel containing the continuous phase solution is arranged as a L-shaped structure.

In some embodiments, the channels containing the containing the discrete phase solution and the continuous phase solution may not be elongated in shape. Instead, these channels may be curved, contain sharp turns at various angles, or spirally shaped.

In some embodiments, the continuous phase solution is flowing or rests in a stationary position on an open surface and is not restricted to any container or channel. For example, the continuous phase solution may be placed on an open surface. Droplets introduced into the continuous phase solution may condense over time or as the droplets flow in the continuous flow solution into silk fibroin particles as the silk fibroin in the discrete phase condenses. Factors such as temperate, fluid forces exerted onto the discrete phase droplets by the continuous phase solution after the droplets have been introduced into the continuous phase solution may affect the rate of condensation in the discrete phase solution.

In some embodiments, the container including the discrete phase solution is an irregularly shaped pouch with an opening to dispense its contents. Such an opening is in contact with the immiscible solution.

In some embodiments, the co-flow device may include multiple channels containing a continuous phase solution, each of which come into contact with the discrete phase solution droplet. The discrete phase solution forms a droplet as it exits its container and passes into the container containing a continuous phase solution due to forces induced on the droplet by the net movement of the continuous phase solution relative to the discrete phase solution. Once such a droplet forms, it flows in the container initially containing the continuous phase solution as a mixture of the continuous phase solution and the discrete phase solution droplets until it exits the continuous phase solution container at a release site (i.e., channel end, pore, opening, etc.). At the release site of the continuous phase container, the mixture comes into contact with a second continuous phase solution. The net movement of the mixture including the discrete phase droplets with the second continuous phase solution induces forces on the mixture, and the droplets, that may might cause the discrete phase droplets to break off into smaller sized droplets. The co-flow device may include multiple such containers of continuous phase solutions that can be used to add an additional degree of control over droplet size, and consequently on the particle size of the silk fibroin particle.

### Additional Embodiments

In any of the embodiments contemplated in the present disclosure, silk fibroin compositions described herein may further comprise additional element(s), including, without limitation, one or more agents, such as biologically active agents. Examples of classes of agents that may be incorporated in a silk fibroin solution include, without limitation, proteins (polypeptides), protein complexes, antigens or immunogens, viral particles and other pathogens, immunoglobulins (antibodies), hormones, cytokines, chemokines, neurotransmitters, pharmacological agonists and antagonists, therapeutic agents (e.g., drugs, antibiotics, etc.), vaccines, toxins, chemical compounds, nutriceuticals, etc.

In some embodiments, provided compositions comprise a vaccine product selected from the group consisting of: Anthrax vaccine (BioThrax); BCG (Bacillus CalmetteGuerin) (Tice, Mycobax); DTaP (Daptacel); DTaP (Infanrix); DTaP (Tripedia); DTaP/Hib (TriHIBit); DTaP-IPV (Kinrix); DTaP-HepB-IPV (Pediarix); DtaP-IPV/Hib (Pentacel); DT (diphtheria vaccine plus tetanus vaccine) (Sanofi); Hib vaccine (ACTHib); DT (Massachusetts); Hib (PedvaxHib); Hib/Hep B (Comvax); Hep A (Havrix), Hepatitis A vaccine; Hep A (Vaqta), Hepatitis A vaccine; Hep B (Engerix-B), Hepatitis B vaccine; Hep B (Recombivax), Hepatitis B vaccine; HepA/HepB vaccine (Twinrix); Human Papillomavirus (HPV) (Gardasil); Influenza vaccine (Afluria); Influenza vaccine (Fluarix); Influenza vaccine (Flulaval); Influenza vaccine (Fluvirin); Influenza vaccine (Fluzone); Influenza vaccine (FluMist); IPV (Ipol), Polio vaccine; Japanese encephalitis vaccine (JEVax); Japanese encephalitis vaccine (Ixiaro); Meningococcal vaccine (Menactra); MMR vaccine (MMR-II); MMRV vaccine (ProQuad); Pneumococcal vaccine (Pneumovax); Pneumococcal vaccine (Prevnar); Poliovirus inactivated (Poliovax), Polio vaccine; Rabies vaccine (Imovax); Rabies vaccine (RabA vert); Rotavirus vaccine (RotaTeq); Rota virus vaccine (Rotarix); Td vaccine (Decavac); Td vaccine (Massachusetts); Tdap vaccine (Adacel); Tdap vaccine (Boostrix); Typhoid (inactivated-Typhim Vi), Typhus vaccine; Typhoid (oral-Ty21a), Typhus vaccine; Vaccinia (ACAM2000); Varicella vaccine (Varivax); Yellow fever vaccine (YF-Vax); Zoster vaccine (Zostavax); and any combinations thereof.

In some embodiments, an agent to be added to a silk fibroin compositions described herein may be selected from the following list of pharmaceuticals/biologics that typically require refrigeration for storage or shipment: Actimmune® (interferon gamma 1B); Amoxil® (amoxicillin); Augmenting (amoxicillin/clavulanic acid); Benzamycin® (erythromycin/benzoyl peroxide); Benzaclin® (clindamycin/benzoyl peroxide); Betaseron® (interferon beta 1B); Caverject® (alprostadil); Ceftin® (cefuroxime axetil); Cefzil® (cefprozil); Cipro® (ciprofloxacin); Combipatch® (estradiol/norethindrone); DDAVP® (desmopressin); Duricef® (cefadroxil); Emcyt® (estramustine phosphate); Enbrel® (etanercept); Epogen® (epoetin alfa); Fortovase® (saquinavir); Genotropin® (somatropin); Humalog® (insulin lispro); Humatrope® (somatropin); Humulin® (insulin); Iletin® (insulin); Infergen® (interferon alfacon-1); Insulin; Kaletra™ (lopinavir/ritonavir); Lantus® (insulin glargine); Leukeran® (chlorambucil); Lorabid® (loracarbef); Miacalcin® (calcitonin salmon); Muse® (alprostadil); Mycostatin® (nystatin); Neupogen® (filgrastim); Norditropin® (somatropin); Norvir® (ritonavir); Novolin® (insulin); Novolog® (insulin aspart); Phenergan® (promethazine); Procrit® (epoetin alfa); Rapamune® (sirolimus); Rebetron™ (ribavirin/interferon alpha 2B); Regranex® (becaplermin); Sandostatin® (octreotide); Suprax® (cefixime); Thyrolar® (liotrix); Trimox® (amoxicillin); Veetids® (penicillin); Velosulin® (insulin), VePesid® (etoposide); Vibramycin® (doxycycline); Viroptic® (trifluridine); Xalatan® (latanoprost); Zithromax® (azithromycin); and any combinations thereof.

In some embodiments, biologically active agents may be or comprise oxygen-binding molecules. In some embodiments, oxygen binding molecules may be or comprise heme-containing moieties (e.g., hemoglobin, myoglobin, neuroglobin, cytoglobin, and/or leghemoglobin).

### Examples

We present here a simple and effective approach for the synthesis of silk fibroin micro- and submicron spheres using a co-flow capillary device where a continuous phase (PVA) is flowed over a discrete phase (silk solution). The device allows the generation of uniform silk spheres without filtering, with simple purification steps, and with the possibility to generate consistently sized micro- and submicron spheres with tunable diameters. Sphere diameter was controlled by varying the concentration of PVA, silk concentration, the flow rate ratios, and silk molecular weight distribution.

Silk micro- and submicron spheres have been previously prepared using spray drying and freeze drying,^{[23-26]} chemical modification by nucleation using eADF4(C16),^{[27,28]} adding organic solvents such as ethanol or methanol in a drop wise fashion,^{[29-31]} water/oil emulsions,^{[32-38]} and blending with immiscible polymers such as poly(vinyl) alcohol (PVA).^{[39]} To date, there has been little study using microfluidic approaches to synthesize uniform microspheres and submicron spheres. Polymers such as alginate, poly(lactic-co-glycolic acid) (PLGA), and poly(L-lactide) (PLA) have been used in microfluidic devices to generate microspheres.^{[11]} A co-flow focusing device was used with silk fibroin and oleic acid to synthesize large microspheres with diameters between 145-200 μm.^{[38]} Such particles may be difficult to purify because of oleic acid residues. Moreover, this synthesis required an additional step to stabilize the particles by physically crosslinking (beta sheet formation) the silk fibroin with methanol, ethanol, or isopropanol.

Without wishing to be bound by a particular theory, sphere generation occurs by break-off of the discrete phase in the bulk fluid stream leading to the formation of monodispersed droplets.^{[9]} Self assembly of silk fibroin in the droplets produces consistently sized spheres in the micron or submicron range. The type of break-off of the droplet is determined by the volumetric flow rates of the continuous and discrete flows of the two immiscible fluids, the interfacial tension, and viscosity of the fluids. This in turn determines the size of the droplets that break-off from the initial stream. Two droplet forming regimes are known in co-flow devices, dripping and jetting.^{[2,9,38]} Dripping is based on the difference between viscous drag forces and the surface tension holding the drop to the bulk fluid stream, while jetting is caused by the Rayleigh-Plateau instability within the inner stream.^{[9,38]} In this case, we hypothesize that silk submicron spheres are generated in the jetting regime. Devices operating in the jetting regime have been shown to generate smaller diameter particles compared to the dimensions of the outlet orifice, in agreement with what is observed here.^{[5,9]} In this case, both fluids are aqueous solutions with low interfacial tension that leads to a reduction in the driving force for liquid jets to break-off into droplets.^{[5]} In the particular case of a coaxial device operating in the narrowing jet regime the equation that predicts the droplet size as function of the fluid parameters is:

$${d}_{d} = a \cdot {( \frac{6 {Q}_{d} {d}_{j}}{{k}^{*} {U}_{c}} )}^{\frac{1}{3}} - b ,$$

where d_{d} is the droplet diameter, a and b are parameters that depend on the device geometry, Q_{d} is the discrete phase flow rate, U_{c} is the continuous phase velocity, dⱼ is the jet diameter, that is a function of (Q_{d}/U_{c}), and k* is a dimensionless wavenumber that is a function of the viscosity ratio between the two fluids k*=k*(η_{d}/η_{c}), with η_{d} is the viscosity of the discrete phase and η_{c} is the viscosity of the continuous phase.^{[3]} Once the droplet of the discrete phase is formed silk will condense because its immiscibility with PVA, following the same mechanism reported previously.^{[39]} With respect to the previous work on the silk spheres formation in a PVA solution bath the possibility to condense the sphere from a droplet of defined and consistent size allows the possibility to produce silk micron- and submicron spheres with better control on the size distribution. Since the droplet diameter can be controlled by changing fluid parameters like flow rate and viscosity, spheres with controlled and tunable size can be synthesized.

In the non-limiting embodiment described below, a coaxial device composed of two dispensing needles to form a concentric system was designed (FIG. 1a). The outer channel consisted of a 16-gauge needle with an inner diameter of 1.2 mm, while the inner channel was a 30-gauge needle with an inner diameter of 152 μm. The resulting device is versatile, reusable, and cost-effective, relying on stainless steel needles that are easily interchangeable and available in many diameters. This improves ease of use with respect to conventional microfluidic devices given the ability to control flow rates in spite of the limitation imposed by the available fixed diameter range of stainless steel luer-lock needles. Additionally, stainless steel can be sterilized without damaging the integrity of the device therefore allowing this system to be used for biological and biomedical applications.

Viscosity measurements were measured using a Brookfield viscometer (Brookfield engineering laboratories, Middleboro, Mass., USA). The viscosity of silk solution depends on its concentration and its molecular weight. A high concentration solution has a dynamic viscosity larger than low concentration solutions. Silk solutions with the same concentration but larger molecular weight (shorter boil time) have higher dynamic viscosities compared to lower molecular weight solutions (longer boil time). Therefore, silk solutions with higher concentrations and shorter boil times have higher dynamic viscosities. Silk solution with concentrations of 60 mg/ml, 30 mg/ml, and 10 mg/ml were measured using two different molecular weights (30 minute boil and 60 minute boil). An up-down rate ramp was conducted on 500 μl of solution with shear rates from 37 to 750 Hz. Three measurements of shear stress were averaged at 5 second increments for individual shear rates. The plastic viscosity was calculated for each solution using the Bingham model for viscoplastic materials defined as τ=τ0+ηD where τ is the shear stress, τ₀ is the yield stress, D is the shear rate, and η is the viscosity. Solving for the slope of the equations provides the dynamic viscosity (η).

Ultimately, the combined control of fluid viscosity and flow rates enables generation of monodisperse silk spheres of tunable diameters.

FIG. 1b-c shows a histogram and a scanning-electron microscope (SEM) image of a typical unfiltered sample of silk nanospheres. The sample was obtained using a continuous phase of 5% PVA at 4 ml/hr flow rate and a discrete phase of 60 mg/ml silk boiled for 60 minutes at 0.4 ml/hr flow rate. The SEM image showed monodisperse, spherical particles with a smooth surface morphology. The distribution was centered at 2410±20 nm as measured by DLS.

The diameter observed with SEM images agrees with DLS measurement implying that there is no shrinkage of the spheres upon drying as shown by other synthesis methods.^{[38]}

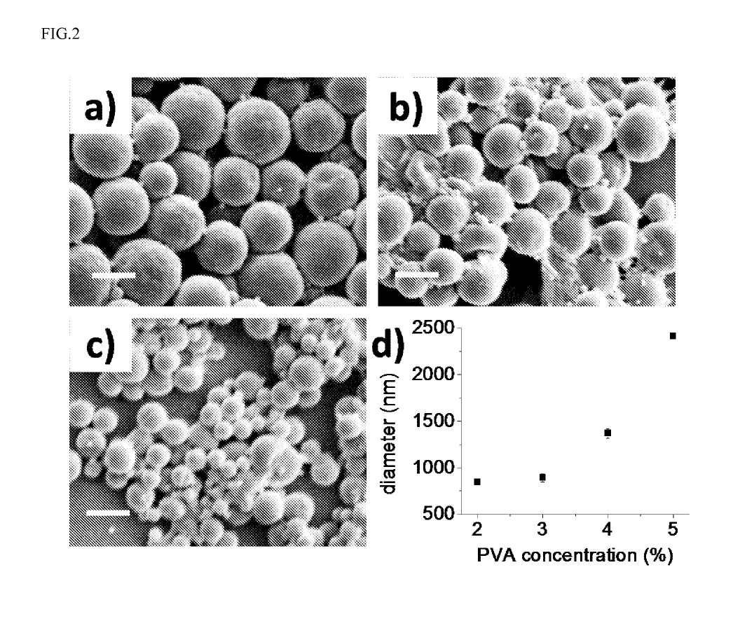

In order to assess the interplay between continuous phase flow and discrete phase flow on the resulting particle diameter, both the concentration of PVA and of silk were independently varied. The viscosity of both PVA and the silk solutions alters the size of the droplet.^{[40]} At first, concentrations of PVA ranging between 2-5% were used to evaluate size variation while maintaining constant both the flow rates (continuous flow rate: Q_{c}=4 ml/hr and discrete flow rate: Q_{d}=0.4 ml/hr) and silk concentration (60 mg/ml). FIG. 2 shows SEM images of the resulting silk spheres as a function of the different PVA concentrations used. The results show that at lower PVA concentrations, smaller spheres were synthesized. Particles with sizes from 850±10 nm to 2410±20 nm were generated as shown in FIG. 2d. The plot shows that microsphere diameter decreased monotonically with a decrease in PVA concentration. The decrease in sphere diameter can be attributed to the viscosity ratio term k*(η_{d}/ηₙ) in the droplet size equation.^{[3]}

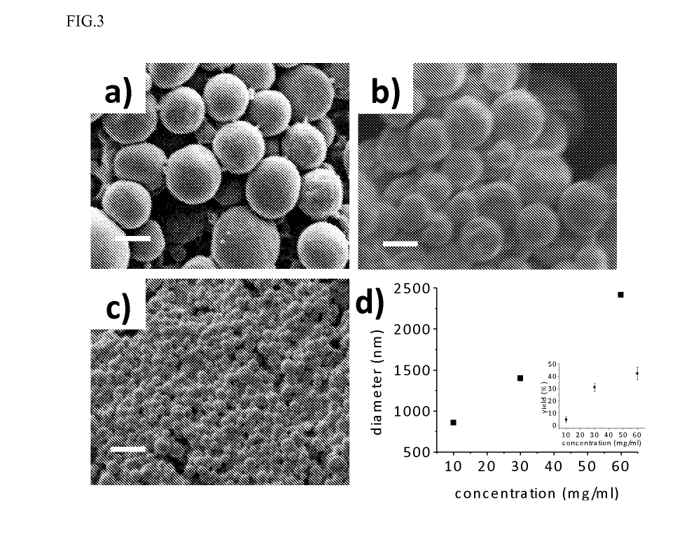

Previous work in similar systems shows that decreasing the concentration of the polymer solution will result in the formation of smaller spheres.^{[8,10]} Here silk solutions of different concentrations were evaluated while keeping continuous and discrete phase flows constant. Silk solutions of 10 mg/ml, 30 mg/ml and 60 mg/ml were fed at a constant flow rate of 0.4 ml/hr into a continuous phase of 5% PVA solution flowing at Q_{c}=4 ml/hr. FIG. 3 shows the resulting spheres indicating a correlation between diameter decrease and decrease in silk concentration. DLS measurements performed on these samples gave sphere diameters of 850±20 nm, 1400±40 nm, and 2410±20 nm for silk concentrations of 10 mg/ml, 30 mg/ml, and 60 mg/ml, respectively as shown in FIG. 3d. The change in sphere diameter was not solely attributable to the reduction in silk mass per droplet. Decreasing the concentration of silk will decrease the solution viscosity and the total mass of silk in the discrete phase. In this case two agonist effects are expected: from the droplet equation a decrease in viscosity of the discrete phase should result in an increased droplet size; from the decreasing in concentration a decrease in mass per droplet and so a decrease in sphere size is expected. From a simple mass balance calculation, assuming only a decrease in silk solution concentration we can define the ratio

$$ρ = \frac{{r}_{1}}{{r}_{2}} = \sqrt[3]{\frac{{m}_{1}}{{m}_{2}}} ,$$

where r₁ and r₂ represent the two silk sphere radii and m₁ and m₂ represent the mass of silk in the different droplets associated with different concentrations. Different spheres with mass ratio of 1/2, 1/3, and 1/6 respectively (associated with 60 mg/ml and 30 mg/ml samples, 30 mg/ml and 10 mg/ml samples, and 60 mg/ml and 10 mg/ml samples) yields values of ρ of 0.794, 0.693, and 0.550 respectively. Measured values from the synthesis, however, did not match these estimates: when decreasing concentration silk spheres were of smaller size than expected from the pure mass balance. This different behavior can be explained by hypothesizing that the diminishing concentration of silk in the discrete phase affects the nucleation efficiency and the formation of new silk spheres.

To corroborate this, the particle yield was measured as a function of different concentrations of silk and the results are presented in the inset of FIG. 3d. The measured yield was found to be 42±5% when using 60 mg/ml, 31±3% when using 30 mg/ml and 5±2% when using 10 mg/ml.

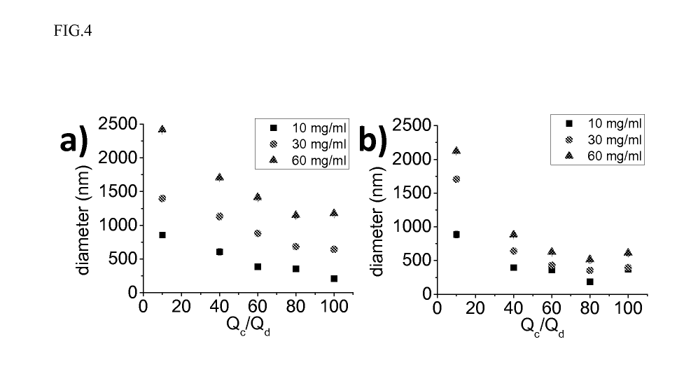

The interplay between continuous and discrete phases can also be modulated by adjusting the flow rate ratio Q_{c}/Q_{d}. For co-flow capillary devices, smaller droplet sizes are obtained as the flow rate ratio increases.^{[2-4,9,38]} Keeping silk and PVA concentrations fixed, the silk flow rate was varied from 0.04 ml/hr to 0.4 ml/hr while Q_{c} was held constant at 4 ml/hr. Flow rate ratios Q_{c}/Q_{d} from 100 to 10 were evaluated yielding particles with sizes ranging from 1170±20 nm to 2410±20 nm (for a silk solution concentration of 60 mg/ml with 60 minute boil time) as illustrated in FIG. 4a. As expected, microsphere diameter decreased with a decrease in the discrete phase flow rate. By reducing the concentration of silk fibroin to 10 mg/ml and 30 mg/ml, it was possible to generate submicron spheres with diameters ranging from 210±20 nm to 860±20, and 640±20 nm to 1400±40 nm, respectively for the same flow rate ratios above, as shown in FIG. 4b.

Increasing the molecular weight of the silk fibroin chains can be achieved by reducing the boiling time of the protein.^{[16,17,22,41]} To determine the effect of molecular weight on sphere synthesis, three boiling times (10, 30 and 60 minutes) were examined, which correspond to an average molecular weight distribution of 400 kDa, 150 kDa, and 50 kDa respectively.^{[41]} As shown in FIG. 4 thirty minute boiled silk with a concentration of 60 mg/ml produced spheres with diameters ranging from 600±40 nm to 2120±20 nm at 100 and 10 flow rate ratios respectively. Sixty minute boiled silk with a concentration of 60 mg/ml produced spheres ranging from 1170±20 nm 2410±20 nm with the same flow rate ratio. Ten min boiled silk solution was unsuccessful because it clogged the inner needle and produced spheres of inconsistent size. From the droplet model, a decrease in the droplet diameter was expected as the viscosity of the discrete phase increases because the k* term in the equation is a function of the viscosity ratio k*=k*(η_{d}/η_{c}). While in the case of silk solution with a concentration of 10 mg/ml a similar viscosity was measured (1.12 cP for 30 minute boiled, 1.25 cP for 60 minute boiled) and particles of similar size were generated over the flow rate ratios used.

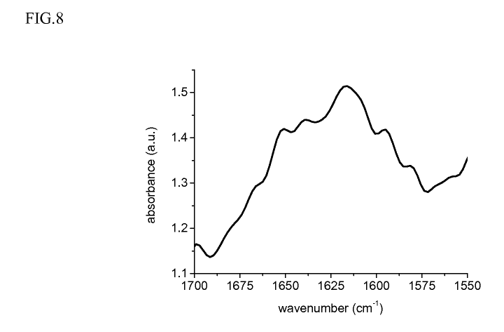

Silk microparticles have been used in the past for drug release applications. In the previous literature, especially in the case of silk films, the control on the degradation kinetics was achieved by controlling the crystallinity of silk. Silk fibroin crystallization leads to the formation of beta-sheet structures that have absorption features in the 1616 cm⁻¹-1637 cm⁻¹ region.^{[42]} To characterize the structure of silk fibroin in the microspheres the amide I absorption band in the 1605 cm⁻¹-1705 cm⁻¹ region was analyzed. Briefly, FTIR characterization was performed using a JASCO FTIR 6200 spectrometer in transmission. The structure of silk fibroin in the microspheres was characterized by analyzing the amide I absorption band of the proteins' secondary structure. Silk fibroin crystallization leads to the formation of beta-sheets structures that have absorption features in the 1616 cm⁻¹-1637 cm⁻¹ region. In the FTIR spectrum (FIG. 8), silk microspheres have an absorption peak in the 1606-1629 cm⁻¹ region which relates to the extensive presence of beta-sheet structure in the particles.

In the case of silk spheres the absorption spectrum (FIG. 8) was overlapped with the scattering of the infrared light which resulted in a shift of about 10 cm⁻¹ to 15 cm⁻¹ to lower wavenumber of the features. This shift was determined using the so called side chain band (1605 cm⁻¹-1615 cm⁻¹) that was observed at 1595 cm⁻¹ in our sample. Because of the scattering, a quantitative analysis of the Amide I band wasn't possible, but from a qualitative point of view silk microspheres showed an intense absorption peak in the 1606 cm⁻¹-1629 cm⁻¹ region which can be related to the presence of beta-sheet structure in the nanoparticles (reference value for the beta-sheet band are 1622 cm⁻¹-1637 cm⁻¹).^{[42]} However in the present work crystallization of silk fibroin was not being used as a mean to control particles' degradation kinetics.

The monodispersity and the tunability of the sphere diameters achievable with the co-flow device, enables its use to directly generate silk-based functional microspheres. The ability to control the sphere diameter is relevant for applications such as controlled release of encapsulated drugs where the size distribution determines the release kinetics of the encapsulated therapeutics.

As a model drug, albumin-fluorescein isothiocyanate conjugate (FITC-BSA) and fluorescein iscothiocyante conjugated dextran (MW=70,000 kDa and MW=2,000-5,000 kDa, respectively) were used. Each model drug was mixed with the silk solution prior to flowing through the co-flow device and generating doped microspheres. To characterize the release profiles, three different sizes of loaded microspheres were examined (1000 nm, 1400 nm, 2410 nm). Loading efficiency was poor (5%) for dextran samples compared to FITC-BSA (95%). Similarly to previously reported studies, this difference is due to the molecular weight and the hydrophobicities of the loading molecules and their interaction with silk.^{[39]}

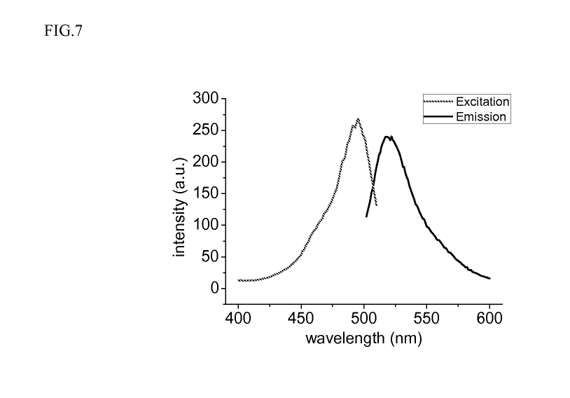

The release of the FITC-BSA model drug over time was evaluated by monitoring the sample fluorescence at λ=524 nm. Briefly, the excitation and emission spectra of spheres loaded with FITC conjugated albumin (FITC-BSA) were measured. A Varian Cary Eclipse Fluorescence Spectrophotometer (Agilent Technologies, Santa Clara, Calif.) was used to measure the fluorescence of the FITC-BSA loaded spheres. The excitation wavelength of the FITC-BSA was measured at 497 nm and the emission wavelength was measured at 524 nm (FIG. 7). No overlap of the excitation/emission spectra of the silk spheres was found with the excitation/emission spectra of FITC-BSA.

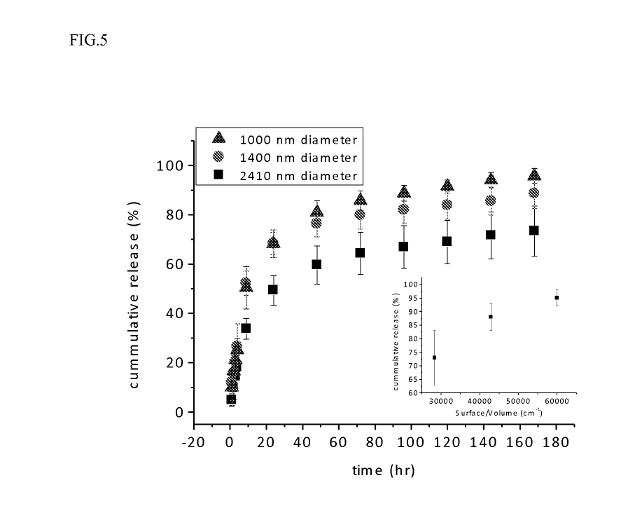

The degradation study was performed in PBS at 37° C. for one week, measuring the fluorescence of supernatant at designated time points (FIG. 5). Release percentage is calculated by the accumulative release measured from the supernatant divided by the total amount of FITC-albumin loaded into the spheres. Following a burst release after the initial 24 hrs (40% of the total), the remaining drug exhibited size-dependent kinetics with spheres of smaller diameter showing faster release. At the 7th day spheres with diameter of 1000 nm released 94% of total loading, while spheres with a diameter of 1400 nm and 2410 nm showed a release of 88% and 73% respectively. The difference in release was found to be nearly linear with the different surface to volume ratio of the spheres (inset of FIG. 5).

In summary, a synthesis method for the controlled formation of dense, monodisperse micro- and submicron spheres of tunable size prepared from silk fibroin using a co-flow capillary device was demonstrated. Tunable microsphere diameter was achieved by adjusting the flow rate ratio, the concentration of the silk protein, the concentration of PVA, and silk molecular weight. Using 60 mg/ml, 60 min boiled silk and a flow rate ratio of 10 it was possible to synthesize spheres greater than 2000 nm in diameter, while using 10 mg/ml, 60 min boiled silk and a flow rate ratio of 100 it was possible to synthesize spheres as small as 210 nm. Spheres were loaded with a model drug and the control over the release kinetics was obtained by changing the spheres' size. This approach can be used to generate tunable micro- and nanospheres for a variety of biological and chemical applications, including encapsulation of medicines for delivery of drugs and as contrast agents for non-invasive sensing.

### Experimental Methods

Silk Processing:

Production of silk fibroin solution was previously described [16]. The purification of silk fibroin from Bombyx mori cocoons initially involves the removal of sericin, by boiling the cocoons in 0.02 M aqueous solution of sodium carbonate for a sufficient time, for example, for about 30 minutes or 60 minutes. The remaining fibroin bundle is washed in deionized water and dried overnight, and then dissolved in 9.3M aqueous lithium bromide at 60° C. for three hours. Dialysis of the solution against deionized water (dialysis cassettes Slide-a-Lyzer, Pierce, MWCO 3.5K) enables the production of 6% w/v silk fibroin solution.

Viscosity Measurements:

Viscometer measurements were measured using a Brookfield viscometer (Brookfield engineering laboratories, Middleboro, Mass., USA). Silk concentrations of 10 mg/ml, 30 mg/ml, and 60 mg/ml solutions were measured for 30 minute and 60 minute boiled silk. An up-down rate ramp was conducted on 500 μl of individual solutions on shear rates from 37 to 750 Hz. Three measurements of shear stress were averaged at 5 second increments for individual shear rates. The plastic viscosity was calculated for each solution using the Bingham model for viscoplastic materials.

Microfluidic Device and Fabrication:

Type 304 reusable stainless steel needles were purchased from McMaster-Carr with luer lock fittings. Luer lock fittings were machined and screwed into machined holders that created the coaxial needle. Silicone tubing attached the needles to two individual syringe pumps from New Era Pump Systems Inc. The continuous phase was a 2% to 5% (w/v) mixture of poly(vinyl alcohol) (PVA) in water (M_{w} 30,000-70,000, 99+% hydrolyzed from Aldrich) and the discrete phase was silk concentrations of 10, 30, and 60 mg/ml. The continuous phase flow rate was kept constant for all experiments (4 ml/hr) while the discrete phase varied from 0.4 ml/hr to 0.04 ml/hr. All solutions were filtered through a 5 μm filter before use.

The solution collected from the device was cast onto a polydimethylsiloxane (PDMS) surface and left to dry for 24 hours. To remove the PVA the dried films were dissolved in ultrapure water at room temperature. The solution was centrifuged at 11,000×g for 10 minutes at 4° C. The supernatant was discarded and the pellet was resuspended in the same volume of ultrapure water and centrifuged again. The final pellet was suspended and stored in 1 ml of ultrapure water at 4° C.

Scanning Electron Microscope Images:

Images of each batch of microspheres were examined under a Zeiss EVO MA 10 (Carl Zeiss SMT, UK) Scanning Electron Microscope (SEM) at 3 keV. Each sample was sputter coated with palladium/gold before imaging.

Dynamic Light Scattering (DLS):

DLS analysis of the silk nanoparticles was previously described.^{[39]} DLS experiments were conducted using a Brookhaven Instrument BI200-SM goniometer (Holtsville, N.Y.) equipped with a diode laser operated at a wavelength of 532 nm. Quantitative analysis of the distribution of relaxation times and corresponding size distributions were obtained using the non-negative least squares: Multiple Pass (NNLS) method.^{[43,44]} To analyze sphere size the size distribution extrapolated by the DLS was fitted with a Gaussian function; the center of the Gaussian was used to estimate the average size and the sigma value to estimate the variance.

Fourier Transform Infrared Spectroscopy (FTIR):

FTIR analysis of microsphere samples were performed in a JASCO FTIR 6200 spectrometer (JASCO, Tokyo, Japan) in transmission. A drop of the microsphere solution was let to dry on a Si wafer. For each sample, 32 scans were coded with a resolution of 4 cm⁻¹, with a wave number range from 400-4000 cm⁻¹. Fourier self-deconvolution of the infrared spectra covering the amide I region (1595-1705 cm⁻¹) was performed by Opus 5.0 software.

Albumin Fluorescein Isothiocyanate Conjugate Release Kinetics:

The release kinetics of FITC-BSA from silk spheres was studied using three different diameter microspheres. Silk solution was loaded with 2% FITC-BSA per mass of silk. Three concentrations of silk solution using unloaded and loaded solutions (60 mg/ml, 30 mg/ml, and 15 mg/ml) were used with a flow rate of 0.4 ml/hr. Samples were purified using the same process as mentioned above and placed into 1.5 ml microvials containing 1 ml pH 7.4 phosphate buffered saline (PBS). All samples were gently agitated at 37° C. for various time periods up to 7 days.

At 1 hr, 2 hrs, 3 hrs, 4 hrs, 9 hrs, 24 hrs, 48 hrs, 72 hrs, 96 hrs, 120 hrs, 144 hrs, and 168 hrs, the sphere suspensions were centrifuged and the supernatant was collected for analysis. The pellet was resuspended in fresh PBS. The samples were assayed using fluorescent measurements (ex=497 nm, em=524 nm) and compared to a standard curve. After the last day, samples were redissolved in 9.3 M LiBr solution to extract the remaining FITC-BSA to measure the total amount of FITC-BSA present.

### REFERENCES

[1] Northrup, M. A. Microfluidics: A few good tricks. Nat. Mater. 2004 3, 282-283.
[2] Christopher, G. F.; Anna, S. L. Microfluidic methods for generating continuous droplet streams. J. Phys. D 2007 40, R319-R336.
[3] Castro-Hernandez, E.; Gundabala, V.; Fernández-Nieves, A.; Gordillo, J. M. Scaling the drop size in coflow experiments. New J. Phys. 2009 11, 075021.
[4] Morimoto, Y.; Kuribayashi-Shigetomi, K.; Takeuchi, S. A hybrid axisymmetric flow-focusing device for monodisperse picoliter droplets. J Micromech. Microengineering 2011, 21.
[5] Cheung Shum, H.; Varnell, J.; Weitz, D. A. Microfluidic fabrication of water-in-water (w/w) jets and emulsions. Biomicrofluidics 2012, 6.
[6] Wang, J.; Zhang, J.; Han, J. Synthesis of crystals and particles by crystallization and polymerization in droplet-based microfluidic devices. Front. Chem. Eng. China 2010, 4, 26-36.
[7] Duncanson, W. J.; Lin T.; Abate A. R.; Seiffert S; Shah R. K.; Weitz D. A. Microfluidic synthesis of advanced microparticles for encapsulation and controlled release. Lab Chip Miniaturisation Chem. Biol. 2012, 12, 2135-2145.
[8] Schneider, T.; Chapman, G. H.; Häfeli, U. O. Effects of chemical and physical parameters in the generation of microspheres by hydrodynamic flow focusing. Colloids Surf. B Biointerfaces 2011, 87, 361-368.
[9] Lee, W.; Walker, L. M.; Anna, S. L. Role of geometry and fluid properties in droplet and thread formation processes in planar flow focusing. Phys. Fluids 2009, 21.
[10] Serra, C. A.; Chang, Z. Microfluidic-assisted synthesis of polymer particles. Chem. Eng. Technol. 2008, 31, 1099-1115.
[11] Xu, Q.; Hashimoto, M.; Dang, T. T.; Hoare, T.; Kohane, D. S.; Whitesides, G. M.; Langer, R.; Anderson, D. G.; David, H. Preparation of monodisperse biodegradable polymer microparticles using a microfluidic flow-focusing device for controlled drug delivery. Small 2009, 5, 1575-1581.
[12] Elzoghby, A. O.; Samy, W. M.; Elgindy, N. A. Protein-based nanocarriers as promising drug and gene delivery systems. J. Control. Release 2012, 161, 38-49.
[13] Häfeli, U. O. Magnetically modulated therapeutic systems. Int. J. Pharm. 2004, 277, 19-24.
[14] Taylor, K. M. L.; Kim, J. S.; Rieter, W. J.; An, H.; Lin, W.; Lin, W. Mesoporous silica nanospheres as highly efficient MRI contrast agents. J. Am. Chem. Soc. 2008, 130, 2154-2155.
[15] Pham, T.; Jackson, J. B.; Halas, N. J.; Lee, T. R. Preparation and characterization of gold nanoshells coated with self-assembled monolayers. Langmuir 2002, 18, 4915-4920.
[16] Rockwood, D. N.; Preda, R. C.; Yücel, T.; Wang, X.; Lovett, M. L.; Kaplan, D. L. Materials fabrication from Bombyx mori silk fibroin. Nat. Protoc. 2011, 6, 1612-1631.
[17] Pritchard, E. M.; Kaplan, D. L. Silk fibroin biomaterials for controlled release drug delivery. Expert Opin. Drug Deliv. 2011, 8, 797-811.
[18] Mathur, A. B.; Gupta, V. Silk fibroin-derived nanoparticles for biomedical applications. Nanomedicine 2010, 5, 807-820.
[19] Mandal, B. B.; Kundu, S. C. Self-assembled silk sericin/poloxamer nanoparticles as nanocarriers of hydrophobic and hydrophilic drugs for targeted delivery. Nanotechnology 2009, 20.
[20] Wang, X.; Wenk, E.; Matsumoto, A.; Meinel, L.; Li, C.; Kaplan, D. L. Silk microspheres for encapsulation and controlled release. J. Control. Release 2007, 117, 360-370.
[21] Gupta, V.; Aseh, A.; Rios, C. N.; Aggarwal, B. B.; Mathur, A. B. Fabrication and characterization of silk fibroin-derived curcumin nanoparticles for cancer therapy. Int. J. Nanomedicine 2009, 4, 115-122.
[22] Pritchard, E. M.; Dennis, P. B.; Omenetto, F.; Naik, R. R.; Kaplan, D. L. Review: Physical and chemical aspects of stabilization of compounds in silk. Biopolymers 2012, 97, 479-498.
[23] Yeo, J.; Lee, K.; Lee, Y.; & Kim, S. Y. Simple preparation and characteristics of silk fibroin microsphere. Eur. Polym. J. 2003, 39, 1195-1199.
[24] Zhao, Z.; Chen, A.; Li, Y., Hu, J.; Liu, X.; Li, J.; Zhang, Y.; Li, G.; Zheng, Z. Fabrication of silk fibroin nanoparticles for controlled drug delivery. J. Nanopart. Res. 2012, 14.
[25] Wang, L.; Xia, Z. R.; Lv, L. L.; Tang, Q.; Li, M. Z. Preparation of silk fibroin microspheres. Adv. Mater. Res. 2011, 236-238, 1902-1905.
[26] Hino, T.; Shimabayashi, S.; Nakai, A. Silk microspheres prepared by spray-drying of an aqueous system. Pharm. Pharmacol. Commun. 2000, 6, 335-339.
[27] Slotta, U. K.; Rammensee, S.; Gorb, S.; Scheibel, T. An engineered spider silk protein forms microspheres. Angew. Chem. Int. Ed. 2008, 47, 4592-4594.
[28] Lammel, A.; Schwab, M.; Slotta, U.; Winter, G.; Scheibel, T. Processing conditions for the formation of spider silk microspheres. Chem Sus Chem 2008, 1, 413-416.
[29] Cao, Z.; Chen, X.; Yao, J.; Huang, L.; Shao, Z. The preparation of regenerated silk fibroin microspheres. Soft Matter 2007, 3, 910-915.
[30] Shi, P.; Goh, J. C. H. Self-assembled silk fibroin particles: Tunable size and appearance. Powder Technol. 2012, 215-216, 85-90.
[31] Wenk, E.; Wandrey, A. J.; Merkle, H. P.; Meinel, L. Silk fibroin spheres as a platform for controlled drug delivery. J. Control Release 2008, 132, 26-34.
[32] Srisuwan, Y.; Srihanam, P.; Baimark, Y. Preparation of silk fibroin microspheres and its application to protein adsorption. J Macromol. Sci. Part A Pure Appl. Chem. 2009, 46, 521-525.
[33] Baimark, Y.; Srihanam, P.; Srisuwan, Y.; Phinyocheep, P. Preparation of porous silk fibroin microparticles by a water-in-oil emulsification-diffusion method. J Appl. Polym. Sci. 2010, 118, 1127-1133.
[34] Xie, R. J.; Wu, H. Y.; Zhu, M. N.; Huang, Y. Y. Preparation and characterization of silk fibroin microspheres. Adv. Mater. Res. 2011, 175-176, 110-115.
[35] Imsombut, T.; Srisa-Ard, M.; Srihanam, P.; Baimark, Y. Preparation of silk fibroin microspheres by emulsificationdiffusion method for controlled release drug delivery applications. E-Polymers 2011.
[36] Park, C.; Lee, S.; Hur, W. Fabrication of fibroin microspheres and hollow spheres. Polymer Korea 2010, 34, 321-325.
[37] Myung, S. J.; Kim, H.; Kim, Y.; Chen, P.; Jin, H. Fluorescent silk fibroin nanoparticles prepared using a reverse microemulsion. Macromol. Res. 2008, 16, 604-608.
[38] Breslauer, D. N.; Muller, S. J.; Lee, L. P. Generation of monodisperse silk microspheres prepared with microfluidics. Biomacromolecules 2010, 11, 643-647.
[39] Wang, X.; Yucel, T.; Lu, Q.; Hu, X.; Kaplan, D. L. Silk nanospheres and microspheres from silk/pva blend films for drug delivery. Biomaterials 2010, 31, 1025-1035.
[40] Nisisako, T.; Torii, T.; Higuchi, T. Novel microreactors for functional polymer beads. Chem. Eng. J. 2004, 101, 23-29.
[41] Wray, L. S.; Hu, X.; Gallego, J.; Georgakoudi, I.; Omenetto, F. G.; Schmidt, D.; Kaplan, D. L. Effect of processing on silk-based biomaterials: Reproducibility and biocompatibility. J. Biomed. Mater. Res. Part B Appl. Biomater. 2011, 99 B, 89-101.
[42] Hu, X.; Kaplan, D.; Cebe, P. Determining beta-sheet crystallinity in fibrous proteins by thermal analysis and infrared spectroscopy. Macromolecules. 2006, 39, 6161-6170.
[43] Hiemenz, P. C.; Rajagopalan, R. in Principles of colloid and surface chemistry. 1997, Marcel Dekker, New York.
[44] Berne, B. J.; Pecora, R., in Dynamic light scattering: with applications to chemistry, biology, and physics 1976, Wiley, New York.

What is claimed is:

1. A silk fibroin sphere having a diameter between approximately 150 nm and approximately 3.0 μm,
wherein the silk fibroin sphere comprises silk fibroin polypeptides having a beta-sheet content of between about 10% and about 60%;
wherein the silk fibroin sphere is untreated with a crosslinking agent; and,
wherein the silk fibroin sphere is essentially free of an immiscible solution.

2. The silk fibroin sphere of claim 1, wherein the immiscible solution is selected from the group consisting of:
water-soluble polyesters and polymer alcohols.

3. The silk fibroin sphere of claim 1, wherein the immiscible solution is selected from the group consisting of:
aliphatic polyesters, semi-aromatic polyesters, and aromatic polyesters.

4. The silk fibroin sphere of claim 1, wherein the immiscible solution is selected from the group consisting of:
Polyglycolide or Polyglycolic acid (PGA); Polylactic acid (PLA); Polycaprolactone (PCL); Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate) (PHBV); Polyethylene terephthalate (PET); Polybutylene terephthalate (PBT); Polytrimethylene terephthalate (PTT); Polyethylene naphthalate (PEN); Poly(lactic-co-glycolic acid) (PLGA); Vectran; and any combination thereof.

5. The silk fibroin sphere of claim 1, wherein the immiscible solution is selected from the group consisting of:
hydroxylated polymers.

6. The silk fibroin sphere of claim 1, wherein the immiscible solution is or comprises Polyvinyl alcohol (PVA).

7. The silk fibroin sphere of claim 1, wherein the crosslinking agent is selected from the group consisting of: methanol, ethanol and isopropanol.

8. The silk fibroin sphere of claim 1, wherein the silk fibroin polypeptides have an average molecular weight of between about 3.5 kDa and about 350 kDa.

9. The silk fibroin sphere of claim 1 or 8, wherein the surface of the silk fibroin sphere is in contact with an immiscible solution.

10. The silk fibroin sphere of claim 9, wherein the immiscible solution has a concentration of between about 1% and 10%.

11. A uniform silk fibroin sphere composition comprising a population of silk fibroin spheres, wherein the population is uniform in that at least 50% of the silk fibroin spheres in the population have diameters within a specified range, wherein the specified range is about 150 nm and about 3.0 μm

12. The uniform silk fibroin sphere composition of claim 11, wherein the specified range is about 150 nm and about 250 nm.

13. The uniform silk fibroin sphere composition of claim 11, wherein the specified range is about 200 nm and about 400 nm.

14. The uniform silk fibroin sphere composition of claim 11, wherein the specified range is about 300 nm and about 500 nm.

15. The uniform silk fibroin sphere composition of claim 11, wherein the specified range is about 400 nm and about 600 nm.

16. The uniform silk fibroin sphere composition of claim 11, wherein the specified range is about 500 nm and about 1000 nm.

17. The uniform silk fibroin sphere composition of claim 11, wherein the specified range is about 600 nm and about 1000 nm.

18. The uniform silk fibroin sphere composition of claim 11, wherein the specified range is about 800 nm and about 1200 nm.

19. The uniform silk fibroin sphere composition of claim 11, wherein the specified range is about 1000 nm and about 2000 nm.

20. The uniform silk fibroin sphere composition of claim 11, wherein the specified range is about 1500 nm and about 2500 nm.

21. The uniform silk fibroin sphere composition of claim 11, wherein the specified range is about 2000 nm and about 3000 nm.

22. An aqueous silk fibroin solution with a predetermined amount of force being exerted thereon,
wherein the aqueous silk fibroin solution has a specified range of viscosity and/or a specified range of concentrations;
wherein the silk fibroin has an average molecular weight of between about 3.5 kDa and about 200 kDa; and,
wherein the predetermined force creates i) a flow; ii) shear stress; or combination thereof, within the aqueous silk fibroin solution.

23. A method for producing a silk fibroin sphere comprising:
providing a silk fibroin solution having a first parameter value;
providing an immiscible solution having a second parameter value; and
introducing the silk fibroin solution into the immiscible solution to produce a monodispersed droplet that results in a silk fibroin sphere having a diameter within a specified range,
wherein the introduction of the silk fibroin solution into the immiscible solution results in a net movement between the silk fibroin solution and the immiscible solution,
wherein the net movement induces a force on the droplet, and
wherein the force affects the diameter of the silk fibroin sphere.

24. The method of claim 23, wherein the parameter associated with the first and second parameter values is selected from the group consisting of viscosity, flow rate, molecular weight, solution boiling duration, and concentration.

25. The method of claim 23, wherein self-assembly of silk fibroin present in the silk fibroin solution in the monodispersed droplet produces the silk fibroin sphere having a diameter within the specified range.

26. The method claim 23, further comprising:
depositing the monodispersed droplet on a substrate to form a condensed silk sphere; and
centrifuging the condensed silk sphere with ultrapure water to remove any immiscible solution in contact with the condensed silk sphere.

27. The method of 23, wherein the force induced by the net movement is imparted onto the silk fibroin solution to pinch off a portion of the silk fibroin solution to produce the droplet.

28. The method of 23, wherein the net movement is the result of the immiscible solution flowing around the introduced silk fibroin solution.

29. The method claim 23, wherein the specified range is about 150 nm and about 3.0 nm.

30. The method claim 23, wherein the specified range is about 150 nm and about 250 nm.

31. The method claim 23, wherein the specified range is about 200 nm and about 400 nm.

32. The method claim 23, wherein the specified range is about 300 nm and about 500 nm.

33. The method claim 23, wherein the specified range is about 400 nm and about 600 nm.

34. The method of claim 23, wherein the immiscible solution is selected from the group consisting of:
Polyglycolide or Polyglycolic acid (PGA); Polylactic acid (PLA); Polycaprolactone (PCL); Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate) (PHBV); Polyethylene terephthalate (PET); Polybutylene terephthalate (PBT); Polytrimethylene terephthalate (PTT); Polyethylene naphthalate (PEN); Poly(lactic-co-glycolic acid) (PLGA); Vectran; and any combination thereof.

35. A method for producing a uniform silk fibroin sphere composition comprising:
providing a silk fibroin having a first parameter value;
providing an immiscible solution having a second parameter value; and
introducing the silk fibroin into the immiscible solution to produce a plurality of monodispersed droplets that result in a plurality of silk fibroin spheres having diameters within a specified range;
wherein the introduction of the silk fibroin solution into the immiscible solution results in a net movement between the silk fibroin solution and the immiscible solution,
wherein the net movement induces a force on each droplet of the monodispersed droplets, and
wherein the force affects the diameter of each silk fibroin sphere of the plurality of silk fibroin spheres.

36. The method of claim 35, wherein the parameter associated with the first and second parameter values is selected from the group consisting of viscosity, flow rate, molecular weight, solution boiling duration, and concentration.

37. The method of claim 35, wherein self-assembly of silk fibroin present in the silk fibroin solution in each of the plurality of monodispersed droplets produces the plurality of silk fibroin spheres having diameters within the specified range.

38. The method claim 35, further comprising:
depositing the plurality of monodispersed droplets on a substrate; and
centrifuging the plurality of monodispersed droplets deposited on a substrate with ultrapure water to remove any immiscible solution in contact with the plurality of silk spheres.

39. The method of 35, wherein the force induced by the net movement is imparted onto the silk fibroin solution to pinch off portions of the silk fibroin solution to produce the plurality of monodispersed droplets.

40. The method of 35, wherein the net movement is the result of the immiscible solution flowing around the introduced silk fibroin solution.

41. The method of claim 35, wherein each monodispersed droplet of the plurality of monodispersed droplets is subject to the same net movement and the same force as other monodispersed droplets used to produce the uniform silk fibroin sphere composition.

42. The method claim 35, wherein the specified range is about 150 nm and about 3.0 μm.

43. The method claim 35, wherein the specified range is about 150 nm and about 250 nm.

44. The method claim 35, wherein the specified range is about 200 nm and about 400 nm.

45. The method claim 35, wherein the specified range is about 300 nm and about 500 nm.

46. The method claim 35, wherein the specified range is about 400 nm and about 600 nm.

47. The method of claim 35, wherein the immiscible solution is selected from the group consisting of:
Polyglycolide or Polyglycolic acid (PGA); Polylactic acid (PLA); Polycaprolactone (PCL); Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate) (PHBV); Polyethylene terephthalate (PET); Polybutylene terephthalate (PBT); Polytrimethylene terephthalate (PTT); Polyethylene naphthalate (PEN); Poly(lactic-co-glycolic acid) (PLGA); Vectran; and any combination thereof.

48. A device for producing a silk fibroin particle comprising:
a container, including a discrete phase solution, having an opening from which a portion of the discrete phase solution is discharged as a droplet; and
a body including a continuous phase solution,
wherein the body is positioned with respect to the container such that the opening of the container has direct access to the continuous phase solution in the body,
wherein a net movement of the continuous phase solution relative to the discrete phase solution at the opening of the container induces a force on the discrete phase solution causing the discrete phase solution to be discharged as a droplet into the body; and
wherein a region of the body includes a mixture of the continuous phase solution and the discrete phase solution discharged from the opening of the container into the body;

49. The device of claim 48, wherein the discrete phase solution is a silk fibroin solution and the continuous phase solution is an immiscible solution selected from the group consisting of:
Polyglycolide or Polyglycolic acid (PGA); Polylactic acid (PLA); Polycaprolactone (PCL); Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate) (PHBV); Polyethylene terephthalate (PET); Polybutylene terephthalate (PBT); Polytrimethylene terephthalate (PTT); Polyethylene naphthalate (PEN); Poly(lactic-co-glycolic acid) (PLGA); Vectran; and any combination thereof.

50. The device of claim 48, wherein the container is at least partially positioned inside the body such that the continuous phase solution surrounds the opening of the container.

51. The device of claim 48, wherein a magnitude of the induced force on the discrete phase solution affects the portion of discrete phase solution discharged in a droplet from the container into the body.

52. The device of claim 48, wherein the portion of discrete phase solution discharged in a droplet affects the size of the silk particle that condenses from the droplet of discrete phase solution.

53. The device of claim 48, wherein the portion of discrete phase solution discharged in a droplet can be controlled by varying at least one of viscosity, flow rate, molecular weight, solution boiling duration, and concentration of at least one of the discrete phase solution of the discrete phase solution and the continuous phase solution.

54. The device of claim 48, wherein silk fibroin in the discrete phase solution self assembles to form a silk fibroin particle.

55. The device of claim 48, further comprising:
a first silicone tubing connected to the container and a first pump containing the discrete phase solution, wherein the first pump delivers the discrete phase solution to the container through the first silicon tubing; and
a second silicone tubing connected to the body and a second pump containing the continuous phase solution, wherein the second pump delivers the continuous phase solution to the body through the second silicon tubing.

56. The device of claim 48, wherein the region of the body including the mixture of the continuous phase solution and the discrete phase solution has direct access to a collection unit and wherein the mixture is transferred onto the collection unit on which the discrete phase solution condenses to form silk particles.

57. The device of claim 56, wherein the silk particles are silk fibroin spheres having diameters within a specified range.
